SEQUENCE LISTING (16 sequences)

SEQ ID NO: 1 - DNA, 62 bp, Artificial Sequence
  Synthetic polynucleotide
ccggcgatggccatggatcatcatcatcatcatcacttacccaatatcaccattttagcaac

SEQ ID NO: 2 - DNA, 42 bp, Artificial Sequence
  Synthetic polynucleotide
attccgatatccatggttagtactgattgaagatctgctgga

SEQ ID NO: 3 - DNA, 61 bp, Artificial Sequence
  Synthetic polynucleotide
ccggcgatggccatggatcatcatcatcatcatcacgctaaaggggaagtgactatcctag

SEQ ID NO: 4 - DNA, 41 bp, Artificial Sequence
  Synthetic polynucleotide
attccgatatccatggttaataggtggagaagatcttttgg

SEQ ID NO: 5 - DNA, 45 bp, Artificial Sequence
  Synthetic polynucleotide
gtgaatatcggctccnnsgacatgaacgataatgtctggctgaca

SEQ ID NO: 6 - DNA, 45 bp, Artificial Sequence
  Synthetic polynucleotide
ggtgttaatttttttcgccagtgtcagccagacattatcgttcat

SEQ ID NO: 7 - DNA, 22 bp, Artificial Sequence
  Synthetic polynucleotide
ggagccgatattcactacctgc

SEQ ID NO: 8 - DNA, 24 bp, Artificial Sequence
  Synthetic polynucleotide
aaaaaaattaacaccgactgcgat

SEQ ID NO: 9 - protein, 326 aa, Escherichia coli
LPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 10 - protein, 326 aa, Artificial Sequence
  Synthetic polypeptide
LPNITILATGGTIAGGGDSATKSNYTVGKVGVENLVNAVPQLKDIANVKGEQVVNIGSLDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 11 - protein, 326 aa, Artificial Sequence
  Synthetic polypeptide
LPNITILATGGTIAGGGDSATKSNYTVGKVGVENLVNAVPQLKDIANVKGEQVVNIGSFDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 12 - protein, 326 aa, Artificial Sequence
  Synthetic polypeptide
LPNITILATGGTIAGGGDSATKSNYTVGKVGVENLVNAVPQLKDIANVKGEQVVNIGSHDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 13 - protein, 348 aa, Wolinella succinogenes
MNAWKKTAVLALMSASVLMAKPQVTILATGGTIAGSGESSVKSSYSAGAVTVDKLLAAVPAINDLATIKGEQISSIGSQEMTGKVWLKLAKRVNELLAQKETEAVIITHGTDTMEETAFFLNLTVKSQKPVVLVGAMRSGSSMSADGPMNLYNAVNVAINKASTNKGVVIVMNDEIHAAREATKLNTTAVNAFASPNTGKIGTVYYGKVEYFTQSVRPHTLASEFDISKIEELPRVDILYAHPDDTDVLVNAALQAGAKGIIHAGMGNGNPFPLTQNALEKAAKSGVVVARSSRVGSGSTTQEAEVDDKKLGFVATESLNPQKARVLLMLALTKTSDREAIQKIFSTY

SEQ ID NO: 14 - protein, 326 aa, Artificial Sequence
  Synthetic polypeptide
LPNITILATGGTIAGGGDSATKSNYTVGKVGVENLVNAVPQLKDIANVKGEQVVNIGGQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 15 - protein, 326 aa, Artificial Sequence
  Synthetic polypeptide
LPNITILATGGTIAGGGDSATKSNYTVGKVGVENLVNAVPQLKDIANVKGEQVVNIGTQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 16 - protein, 326 aa, Artificial Sequence
  Synthetic polypeptide
LPNITILATGGTIAGGGDSATKSNYTVGKVGVENLVNAVPQLKDIANVKGEQVVNIGAQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

(19) United States
(12) Patent Application Publication
(10) Pub. No.: US 2016/0213759 A1
(43) Pub. Date: Jul. 28, 2016

(54) THERAPEUTIC ASPARAGINASES

(71) Applicants: BOARD OF REGENTS, THE UNIVERSITY OF TEXAS SYSTEM, Austin, TX (US); SANDIA CORPORATION, Albuquerque, NM (US); UNIVERSITY OF MARYLAND, College Park, MD (US)

(72) Inventors: Susan REMPE, Albuquerque, NM (US); David M. ROGERS, Tampa, FL (US); Andriy ANISHKIN, Austin, TX (US); Sergei SUKHAREV, Bethesda, MD (US); Philip L. LORENZI, Pearland, TX (US); Wai Kin CHAN, Houston, TX (US); John N. WEINSTEIN, Bellaire, TX (US)

(21) Appl. No.: 14/917,934

(22) PCT Filed: Sep. 10, 2014
(86) PCT No.: PCT/US2014/054977

§ 371 Date: Mar. 9, 2016

Related U.S. Application Data
(60) Provisional application No. 61/876,106, filed on Sep. 10, 2013.

(51) Int. Cl. A61K 38/50; C12N 9/96; C12N 9/82; A61K 47/48; A61K 45/06

(52) U.S. Cl. CPC A61K 38/50; A61K 47/48215; A61K 45/06; C12N 9/82; C12N 9/96; C12Y 305/01001

(57) ABSTRACT

Provided herein are mutant asparaginase enzymes that lack glutaminase activity. Also provided are methods of treating ASNS-negative cancer cells with a glutaminase-free asparaginase.

The present application claims the priority benefit of U.S. provisional application No. 61/876,106, filed Sep. 10, 2013, the entire contents of which is incorporated herein by reference.

The invention was made with government support under Grant No. DE-AC04-94AL85000 awarded by the Department of Energy. The government has certain rights in the invention.

### BACKGROUND OF THE INVENTION

1. Field of the Invention

The present invention relates generally to the fields of medicine and biology. More particularly, it concerns compositions and methods for the treatment of cancer with enzymes that deplete asparagine. Even more particularly, it concerns the engineering of an enzyme with high asparagine degrading activity and low glutamine degrading activity.

2. Description of Related Art

L-Asparaginase (L-ASP) is an enzyme drug used in combination with vincristine and a glucocorticoid (e.g., dexamethasone) to treat acute lymphoblastic leukemia (ALL) (Szymanski et al., 2012; Ortega et al., 1977). A previous study used the Immune Epitope Database consensus method to predict the region of the L-ASP protein sequence that could be reengineered to reduce MHC-II binding without affecting its catalytic and pharmacological properties (Cantor et al., 2011). A rationale for testing L-ASP against low-asparagine synthetase (ASNS) solid tumors has been reported (Bussey et al., 2006; Lorenzi et al., 2006; Lorenzi et al., 2008; Scherf et al., 2000; Dufour et al., 2012). L-ASP's primary known enzymatic activity is deamidation of asparagine to aspartic acid and ammonia, but it also deamidates glutamine to glutamic acid and ammonia, although with lower affinity and lower maximal rate. L-ASP therapy is often limited by toxic side effects that are generally attributed to the glutaminase activity (Warrell et al., 1982; Kafkewitz and Bendich, 1983). Those side effects often preclude completion of the full treatment regimen, resulting in poor outcome (Silverman et al., 2001). However, it is not known whether the therapeutic index of L-ASP would be increased by decreasing its glutaminase activity (Warrell et al., 1982; Kafkewitz and Bendich, 1983) without also decreasing the enzyme's anticancer effect.

### SUMMARY OF THE INVENTION

The present invention concerns the engineering of the E. coli L-Asparaginase II (L-ASP) enzyme such that the modified enzyme retains asparaginase activity but has reduced glutaminase activity relative to the wild-type enzyme, and providing the modified L-ASP enzymes in a formulation suitable for human cancer therapy. To develop such an enzyme, molecular dynamics (MD) simulations of the clinically used Escherichia coli L-ASP were used to guide rational engineering of a glutaminase-deficient variant. Residues that preferentially interacted with glutamine over asparagine, but were not essential to the enzymatic conversion, were chosen as candidates for saturation mutagenesis. The top candidate was amino acid Q59. Modifications of this residue, as well as residue S58, resulted in an enzyme having reduced glutaminase activity. As such, L-ASP enzymes modified as described herein overcome a major deficiency in the art by providing novel enzymes that comprise asparaginase activity while having reduced glutaminase activity as compared to the native enzyme. As such, these modified enzymes may be suitable for cancer therapy, especially for ASNS-deficient cancer.

Accordingly, in a first embodiment there is provided a modified polypeptide, particularly an enzyme variant with asparaginase degrading activity derived from bacterial L-ASP enzymes. For example, a novel enzyme variant may have an amino acid sequence selected from the group consisting of SEQ ID NOs: 10-12 and 14-16. For example, the variant may be derived from a bacterial enzyme, such as E. coli L-Asparaginase II. In certain aspects, there may be a polypeptide comprising a modified L-ASP capable of degrading asparagine but not glutamine. In some embodiments, the polypeptide may be capable of degrading asparagine under physiological conditions. For example, the polypeptide may have a catalytic efficiency for asparagine (k_{cat}/K_{M}) of at least or about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10⁴, 10⁵, 10⁶ s⁻¹M⁻¹ or any range derivable therein.

An unmodified polypeptide may be a native L-ASP, particularly an E. coli isoform or other bacterial isoform. For example, the native E. coli L-ASP may have the sequence of SEQ ID NO: 9. A non-limiting example of another native bacterial L-ASP is W. succinogenes L-ASP (Genbank ID: NP_906890.1; SEQ ID NO: 13). Exemplary native polypeptides include a sequence having about, at least 60%, 65%, 70%, 75%, 80%, 85%, 90%, 95%, 96%, 97%, 98%, 99% or 100% identity (or any range derivable therein) to SEQ ID NOs: 9 or 13 or a fragment thereof. For example, the native polypeptide may comprise at least or up to about 10, 20, 30, 40, 50, 60, 70, 80, 90, 100, 150, 200, 250, 300, 350, 400, 405 residues (or any range derivable therein) of the sequence of SEQ ID NOs: 9 or 13.

In some embodiments, the native L-ASP may be modified by one or more other modifications, such as chemical modifications, substitutions, insertions, deletions, and/or truncations. In a particular embodiment, the native L-ASP may be modified by substitutions. For example, the number of substitutions may be one, two, three, four or more. In further embodiments, the native L-ASP may be modified in the substrate recognition site or any location that may affect substrate specificity. For example, the modified polypeptide may have the at least one amino acid substitution at an amino acid position corresponding to amino acid position 58 or 59 of SEQ ID NO: 9. In these examples, the first amino acid of each sequence corresponds to amino acid position 1, and each amino acid is numbered sequentially therefrom.

In certain embodiments, the substitution at amino acid position 58 is glycine (Gly; G), threonine (Thr; T), or alanine (Ala; A). In a particular embodiment, the substitution may comprise the 58G substitution. In another particular embodiment, the substitution may comprise the 58T substitution. In another particular embodiment, the substitution may comprise the 58A substitution.

In certain embodiments, the substitution at amino acid position 59 is leucine (Leu; L), phenylalanine (Phe; F), or histidine (His; H). In a particular embodiment, the substitution may comprise the 59L substitution. In another particular embodiment, the substitution may comprise the 59F substitution. In another particular embodiment, the substitution may comprise the 59H substitution.

In some embodiments, the native L-ASP may be an E. coli L-ASP. In a particular embodiment, the substitution is a S58G of E. coli L-ASP (for example, the modified polypeptide having the amino acid sequence of SEQ ID NO: 14, a fragment or homolog thereof). In a particular embodiment, the substitution is a S58T of E. coli L-ASP (for example, the modified polypeptide having the amino acid sequence of SEQ ID NO: 15, a fragment or homolog thereof). In a particular embodiment, the substitution is a S58A of E. coli L-ASP (for example, the modified polypeptide having the amino acid sequence of SEQ ID NO: 16, a fragment or homolog thereof). In a particular embodiment, the substitution is a Q59L of E. coli L-ASP (for example, the modified polypeptide having the amino acid sequence of SEQ ID NO: 10, a fragment or homolog thereof). In a particular embodiment, the substitution is a Q59F of E. coli L-ASP (for example, the modified polypeptide having the amino acid sequence of SEQ ID NO: 11, a fragment or homolog thereof). In a particular embodiment, the substitution is a Q59H of E. coli L-ASP (for example, the modified polypeptide having the amino acid sequence of SEQ ID NO: 12, a fragment or homolog thereof).

A modified polypeptide as discussed above may be characterized as having a certain percentage of identity as compared to an unmodified polypeptide (e.g., a native polypeptide) or to any polypeptide sequence disclosed herein. For example, the unmodified polypeptide may comprise at least or up to about 10, 20, 30, 40, 50, 60, 70, 80, 90, 100, 150, 200, 250, 300, 350, 400, 405 residues (or any range derivable therein) of a native bacterial L-ASP (i.e., E. coli or W. succinogenes L-ASP). The percentage identity may be about, at least 60%, 65%, 70%, 75%, 80%, 85%, 90%, 95%, 96%, 97%, 98%, 99% or 100% (or any range derivable therein) between the unmodified portions of a modified polypeptide (i.e., the sequence of the modified polypeptide excluding any substitution at amino acid 58 or 59) and the corresponding native polypeptide. It is also contemplated that percentage of identity discussed above may relate to a particular modified region of a polypeptide as compared to an unmodified region of a polypeptide. For instance, a polypeptide may contain a modified or mutant substrate recognition site of L-ASP that can be characterized based on the identity of the amino acid sequence of the modified or mutant substrate recognition site of L-ASP to that of an unmodified or mutant L-ASP from the same species or across species. For example, a modified or mutant E. coli polypeptide characterized as having at least 90% identity to an unmodified L-ASP means that at least 90% of the amino acids in that modified or mutant E. coli polypeptide are identical to the amino acids in the unmodified polypeptide.

In some aspects, the present invention also contemplates polypeptides comprising the modified L-ASP linked to a heterologous amino acid sequence. For example, the modified L-ASP may be linked to the heterologous amino acid sequence as a fusion protein. In a particular embodiment, the modified L-ASP may be linked to amino acid sequences, such as an IgG Fc, albumin, an albumin binding peptide, or an XTEN polypeptide for increasing the in vivo half-life.

To increase serum stability, the modified L-ASP may be linked to one or more polyether molecules. In a particular embodiment, the polyether may be polyethylene glycol (PEG). The modified polypeptide may be linked to PEG via specific amino acid residues, such as lysine or cysteine. For therapeutic administration, such a polypeptide comprising the modified L-ASP may be dispersed in a pharmaceutically acceptable carrier.

In one aspect, the modified L-ASP may be contained within or on a red blood cell that has been altered so as to comprise (e.g., encapsulate) high levels of an asparaginase. The altered red blood cell may then be used to treat a subject (e.g., human patient), with the introduction of a population of these altered RBCs into the subject to supply the therapeutic enzyme.

In some aspects, a nucleic acid encoding such a modified L-ASP is contemplated. In one aspect, the nucleic acid has been codon optimized for expression in bacteria. In particular embodiments, the bacteria is E. coli. In other aspects, the nucleic acid has been codon optimized for expression in a fungus (e.g., yeast), in insect cells, or in mammalian cells. The present invention further contemplates vectors, such as expression vectors, containing such nucleic acids. In particular embodiments, the nucleic acid encoding the modified L-ASP is operably linked to a promoter, including but not limited to heterologous promoters. In one embodiment, a modified L-ASP may be delivered to a target cell by a vector (e.g., a gene therapy vector). Such vectors may have been modified by recombinant DNA technology to enable the expression of the modified L-ASP-encoding nucleic acid in the target cell. These vectors may be derived from vectors of non-viral (e.g., plasmids) or viral (e.g., adenovirus, adeno-associated virus, retrovirus, lentivirus, herpes virus, or vaccinia virus) origin. Non-viral vectors are preferably complexed with agents to facilitate the entry of the DNA across the cellular membrane. Examples of such non-viral vector complexes include the formulation with polycationic agents which facilitate the condensation of the DNA and lipid-based delivery systems. An example of a lipid-based delivery system would include liposome based delivery of nucleic acids.

In still further aspects, the present invention further contemplates host cells comprising such vectors. The host cells may be bacteria (e.g., E. coli), fungal cells (e.g., yeast), insect cells, or mammalian cells.

In some embodiments, the vectors are introduced into host cells for expressing the modified L-ASP. The proteins may be expressed in any suitable manner. In one embodiment, the proteins are expressed in a host cell such that the protein is glycosylated. In another embodiment, the proteins are expressed in a host cell such that the protein is aglycosylated.

In some embodiments, the enzymes or nucleic acids are in a pharmaceutical formulation comprising a pharmaceutically acceptable carrier. The enzyme may be a native bacterial L-ASP polypeptide or a modified L-ASP polypeptide. The nucleic acid may encode a native bacterial L-ASP polypeptide or a modified L-ASP polypeptide.

Certain aspects of the present invention also contemplate methods of treatment by the administration of the modified L-ASP polypeptide, the nucleic acid encoding the modified L-ASP in a gene therapy vector, or the formulation of the present invention, and in particular methods of treating tumor cells or subjects with cancer. The subject may be any animal, such as a mouse. For example, the subject may be a mammal, particularly a primate, and more particularly a human patient. In some embodiments, the method may comprise selecting a patient with cancer, particularly a patient with an asparagine synthetase (ASNS)-deficient cancer.

In some embodiments, the cancer is any cancer that is sensitive to asparagine depletion. In one embodiment, the present invention contemplates a method of treating a tumor cell or a cancer patient comprising administering a formulation comprising a L-ASP enzyme. In some embodiments, the administration occurs under conditions such that at least a portion of the cells of the cancer are killed. In another embodiment, the formulation comprises such a modified L-ASP lacking glutaminase degrading activity at physiological conditions and further comprising an attached polyethylene glycol chain. In some embodiments, the formulation is a pharmaceutical formulation comprising any of the above discussed L-ASP variants and pharmaceutically acceptable excipients. Such pharmaceutically acceptable excipients are well known to those of skill in the art. All of the above L-ASP variants may be contemplated as useful for human therapy.

In a further embodiment, there may also be provided a method of treating a tumor cell comprising administering a formulation comprising a bacterial (e.g., E. coli) modified L-ASP that has asparagine degrading activity but not glutamine degrading activity, or a nucleic acid encoding thereof.

Because tumor cells are dependent upon their nutrient medium for L-asparagine, the administration or treatment may be directed to the nutrient source for the cells, and not necessarily the cells themselves. Therefore, in an in vivo application, treating a tumor cell includes contacting the nutrient medium for a population of tumor cells with the engineered (i.e., modified) L-ASP. In this embodiment, the medium can be blood, lymphatic fluid, spinal fluid and the like bodily fluid where asparagine depletion is desired.

In accordance with certain aspects of the present invention, such a formulation containing the modified L-ASP can be administered intravenously, intradermally, intraarterially, intraperitoneally, intralesionally, intracranially, intraarticularly, intraprostaticaly, intrapleurally, intrasynovially, intratracheally, intranasally, intravitreally, intravaginally, intrarectally, intratumorally, intramuscularly, subcutaneously, subconjunctival, intravesicularlly, mucosally, intrapericardially, intraumbilically, intraocularly, orally, topically, by inhalation, infusion, continuous infusion, localized perfusion, via a catheter, via a lavage, in lipid compositions (e.g., liposomes), or by other method or any combination of the forgoing as would be known to one of ordinary skill in the art.

In a further embodiment, the method may also comprise administering at least a second anticancer therapy to the subject. The second anticancer therapy may be a surgical therapy, chemotherapy, radiation therapy, cryotherapy, hormone therapy, immunotherapy, or cytokine therapy.

In one embodiment, a composition comprising a modified L-ASP or a nucleic acid encoding a modified L-ASP is provided for use in the treatment of a tumor in a subject. In another embodiment, the use of a modified L-ASP or a nucleic acid encoding a modified L-ASP in the manufacture of a medicament for the treatment of a tumor is provided. Said modified L-ASP may be any modified L-ASP of the embodiments.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding,” with reference to a nucleic acid, are used to make the invention readily understandable by the skilled artisan; however, these terms may be used interchangeably with “comprise” or “comprising,” respectively.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. Distinct coordinations of asparagine and glutamine in the catalytic site of E. coli L-ASP. Snapshots were taken at ˜20 ns of simulation. Q59 typically interacts with the backbone of both asparagine (A) and glutamine (B), but the patterns differ. Asparagine is usually coordinated through its backbone —NH group by the side-chain oxygen of Q59, whereas the backbone carboxyl of glutamine often interacts with the backbone —NH group of Q59, while the side chain of Q59 faces away from the substrate.

FIGS. 2A-F. Enzymatic characterization of Q59 L-ASP mutants. (A) Coomassie blue-stained SDS-PAGE showing expression of L-ASP WT and Q59 mutants. The expression vector was transformed into the E. coli BL-21 strain, and 20 μL of culture supernatant was analyzed by SDS-PAGE. The empty expression vector (Ctrl) and T89V (inactive mutant) served as negative controls for assays of enzyme activity in panels (B) and (C). (B) Asparaginase activity of Q59 mutants by colorimetric assay. (C) Glutaminase activity of Q59 mutants by colorimetric assay. (D) Asparaginase-specific activity of purified Q59 mutants by the colorimetric assay. (E) Glutaminase-specific activity of purified Q59 mutants by the colorimetric assay. (F) Ratio of glutaminase- and asparaginase-specific activities for purified L-ASP mutants. SDS-PAGE, sodium dodecyl sulfate-polyacrylamide gel electrophoresis.

FIGS. 3A-B. Asparagine and glutamine deamidation kinetics. WT L-ASP or Q59L L-ASP was added to a reaction solution containing 100 μM asparagine, 1600 μM glutamine, or both. Concentrations of asparagine (A) and glutamic acid (B) were measured over a 1500-s time series by LC-MS/MS. Solid symbols represent concentrations in the single substrate reaction, and open symbols represent concentrations in the mixture.

FIGS. 4A-L. Anticancer activity of WT, Q59L, and Q59F L-ASP. (A-H) Two ovarian cancer cell lines (OVCAR-8 and SK-OV-3) and six leukemia cell lines (MOLT-4, K562, NALM-6, REH, SR, and CCRF-CEM) were seeded in 96-well plates, incubated for 48 h, treated with a range of (WT, Q59L, or Q59F) L-ASP concentrations for 48 h, and finally assayed with CELLTITER-BLUE® using fluorescence excitation at 544 nm and emission at 590 nm. (I-J) MOLT-4 and OVCAR-8 cells were seeded in 96-well plates and incubated for 48 h, then treated with the indicated concentrations of E. coli L-ASP WT or Q59 mutant for 48 h. Inhibition of cell viability was measured as in panels (A-H). Sham treatment was used as a control. (K) Western blot analysis of ASNS levels in the indicated cells treated with an EC₅₀ dose of L-ASP. (L) Western blot analysis of ASNS levels in OVCAR-8 cells treated with L-ASP mutants. Numbers below the blot represent the relative level of ASNS, which was normalized to the level of the loading control β-actin (set to “1” for the control).

FIGS. 5A-E. Selective growth inhibition of ASNS-negative cancer cells by WT, Q59L, and Q59F L-ASP. (A) Western blot analysis of ASNS levels in OVCAR-8 cells 48 h after transfection with ASNS siRNA (siASNS) or negative control siRNA (siNeg). β-actin was used as a loading control. (B) WT and Q59L L-ASP concentration-activity curves. The OVCAR-8 cell line was transfected with negative control siRNA (siNeg) or ASNS siRNA (siASNS) for 48 h, then treated with a range of L-ASP concentrations for 48 h, and finally assayed with CELLTITER-BLUE®. WT, Q59L, and Q59F L-ASP concentration-activity curves were determined in the (C) Sup-B15 and (D) RS4;11 leukemia cell lines by CELLTITER-BLUE® assay. (E) Western blot analysis of ASNS levels in Sup-B15 and RS4;11 cells treated with an EC₅₀ dose of L-ASP. No treatment was used as a primary control, and MOLT-4 cells were included as a secondary control.

FIGS. 6A-C. Proposed model for the anticancer mechanism of WT and Q59L L-ASP. The mechanism of anticancer activity depends on L-ASP glutaminase activity and ASNS expression. For simplicity, glutamine synthesis pathways are not shown. (A) (Left panel) (1) Q59L L-ASP effectively depletes Asn but not Gln, which (2) is imported by the cancer cell for (3) synthesis of Asn by ASNS, thereby promoting cancer cell proliferation (4). Numbering is omitted from subsequent panels, but analogous interpretation illustrates that the added glutaminase activity of WT L-ASP decreases the extracellular supply of Gln, thereby limiting cancer cell proliferation (right panel). (B) Low-ASNS cancer cells are insensitive to Q59L L-ASP (left panel), but not to WT L-ASP (right panel). (C) ASNS-negative cancer cells are sensitive to both Q59L (left panel) and WT (right panel). Details of the model are provided in the text.

FIG. 7. Expression of L-ASP in bacterial culture medium. Expression vectors of L-ASP wild-type (WT) or an empty vector (Ctrl) were transformed into the expression host BL-21 strain and expression was induced for 4 or 24 h. 20 μL of supernatant and pellet were subjected to SDS-PAGE analysis as described in FIG. 2A. IPTG, β-D-1-thiogalactopyranoside (for induction of L-ASP secretion by the transformed cells). The nominal molecular weight of His-tagged L-ASP is 37 kDa.

FIGS. 8A-D. Optimization of asparaginase and glutaminase assay conditions in bacterial culture supernatants. (A) Standard curve of absorbance at 705 nm as a function of enzyme activity (IU) relative to ELSPAR® in the asparaginase colorimetric assay. The log of the activity was taken to make the graph linear. (B) A standard curve shows the corresponding absorbance at 450 nm to different amounts of glutamate in the glutaminase colorimetric assay. The asparaginase (C) or glutaminase (D) colorimetric assay was performed on the serial dilutions of the L-ASP WT expressing culture supernatant.

FIGS. 9A-C. Comparison of asparaginase and glutaminase activities of W. succinogenes L-ASP and E. coli L-ASP. (A) LC-MS/MS measurement of the product aspartic acid in the enzyme reactions of L-ASP variants using asparagine as a substrate. 10 nM WT, 20 nM Q59L and 160 nM W. succinogenes L-ASP were used in reactions. (B) LC-MS/MS measurement of the product glutamic acid in the enzyme reactions of L-ASP variants using glutamine as a substrate. 40 nM WT, 80 nM Q59L and 640 nM W. succinogenes L-ASP were used in reactions. (C) Ratio of glutaminase-specific activity to asparaginase-specific activity of L-ASP variants. Specific activities (glutaminase and asparaginase) of enzymes are equal to the appearance rate of products [calculated from (A) and (B)] divided by the amount of enzyme used in the reaction.

FIGS. 10A-D. Kinetic analysis of asparaginase and glutaminase activities of Q59L and Q59H L-ASP. (A) Asparaginase colorimetric assay. Enzyme amount was equivalent to 2.5×10⁻³ IU of asparaginase in each 50 μL reaction, and substrate amount was 5 mM. (B) Glutaminase colorimetric assay. Enzyme amount was equivalent to 0.2 IU of asparaginase in each 200 μL reaction, and substrate amount was 200 μM. (C) LC-MS/MS measurement of the product aspartic acid in the enzyme reactions of L-ASP variants using asparagine as a substrate. 10 nM WT, 20 nM Q59L, and 60 nM Q59H were used in reactions. (D) LC-MS/MS measurement of the product glutamic acid in the enzyme reactions of L-ASP variants using glutamine as a substrate. 40 nM WT, 80 nM Q59L, and 240 nM Q59H were used in reactions. Purified enzymes were used in all of reactions.

FIGS. 11A-B. Asparagine and glutamine deamidation kinetics. WT L-ASP was added to a reaction solution containing 1 mM asparagine, 1 mM glutamine, or both. Concentrations of reaction products asparate (A) and glutamate (B) were measured over a time series by LC-MS/MS. Black bars represent measured absolute concentrations in the single substrate reaction, and gray bars represent measured absolute concentrations in the mixture that contained both substrates.

FIG. 12. Anticancer activity of W. succinogenes L-ASP. OVCAR-8 cells were treated with the indicated concentrations of E. coli WT L-ASP, E. coli Q59L L-ASP, and W. succinogenes L-ASP for 48 h. Cell viability was measured with MTS assay with absorbance at 490 nm. Vehicle treatment (0 U/mL) was used as a control.

FIG. 13. Anticancer activity of WT, Q59L, and Q59H L-ASP. Twelve cancer cell lines were treated with a range of WT (solid squares), Q59L (open squares), or Q59H (solid circles) L-ASP concentrations for 48 h then assayed with CELLTITER-BLUE®. The abbreviation in parentheses after each cell line name denotes tissue-of-origin (BR=breast, CO=colon, ME=melanoma, OV=ovarian, PR=prostate, RE=renal).

FIGS. 14A-B. Anticancer activity of Q59L L-ASP in an acute lymphoblastic leukemia mouse model. NOD/SCID/IL2Rgamma knockout (NSG) mice injected with Sup-B15/luciferase cells were treated with PBS or Q59L L-ASP starting two weeks after injection (i.p. 3 times a week for 3 weeks). *Day 0 was the first day of treatment. Luciferase activity of Q59L-treated and control mice was measured over the course of the experiment. (A) Bioluminescent signal from individual mice. (B) Averaged bioluminescence of PBS- and Q59L-treated mice. (C) Representative images illustrating that Q59L treatment prevented leukemia infiltration of the spleen. The left image is a spleen from a mouse treated with PBS. The right image is a spleen from a mouse treated with Q59L L-ASP.

FIGS. 15A-B. Enzymatic characterization of S58 L-ASP mutants. (A) Asparaginase activity of S58 mutants by colorimetric assay. (B) Glutaminase activity of S58 mutants by colorimetric assay.

FIG. 16. Anticancer activity of S58G and S58T L-ASP. The growth inhibitory activity of L-ASP mutants S58G and S58T was tested using Sup-B15 leukemia cells as assayed with CELLTITER-BLUE® using fluorescence excitation at 544 nm and emission at 590 nm.

### DESCRIPTION OF ILLUSTRATIVE EMBODIMENTS

L-Asparaginase (L-ASP) is a key component of therapy for acute lymphoblastic leukemia. Its mechanism of action, however, is poorly understood, in part because of its dual asparaginase and glutaminase activities. Here, L-ASP's glutaminase activity was found to not always be required for the enzyme's anticancer effect. Molecular dynamics simulations of the clinically standard Escherichia coli L-ASP were used to predict what mutated forms could be engineered to retain activity against asparagine but not glutamine. Dynamic mapping of enzyme substrate contacts identified S58 and Q59 as promising mutagenesis targets for that purpose. Saturation mutagenesis followed by enzymatic screening identified S586, S58T, S58A, Q59L, and Q59F as variants that retain asparaginase activity but show low or undetectable glutaminase activity. Unlike wild-type L-ASP, Q59L is inactive against cancer cells that express measurable asparagine synthetase (ASNS). Q59L is potently active, however, against ASNS-negative cells. Thus, the glutaminase activity of L-ASP is necessary for anticancer activity against ASNS-positive cell types but not ASNS-negative cell types. Because the clinical toxicity of L-ASP is thought to stem from its glutaminase activity, these findings suggest that glutaminase-negative variants of L-ASP may provide larger therapeutic indices than wild-type L-ASP for ASNS-negative cancers.

### I. DEFINITIONS

As used herein the terms “protein” and “polypeptide” refer to compounds comprising amino acids joined via peptide bonds and are used interchangeably.

As used herein, the term “fusion protein” refers to a chimeric protein containing proteins or protein fragments operably linked in a non-native way.

As used herein, the term “half-life” (½-life) refers to the time that would be required for the concentration of a polypeptide thereof to fall by half in vitro or in vivo, for example, after injection in a mammal.

The terms “in operable combination,” “in operable order,” and “operably linked” refer to a linkage wherein the components so described are in a relationship permitting them to function in their intended manner, for example, a linkage of nucleic acid sequences in such a manner that a nucleic acid molecule capable of directing the transcription of a given gene and/or the synthesis of desired protein molecule, or a linkage of amino acid sequences in such a manner so that a fusion protein is produced.

The term “linker” is meant to refer to a compound or moiety that acts as a molecular bridge to operably link two different molecules, wherein one portion of the linker is operably linked to a first molecule, and wherein another portion of the linker is operably linked to a second molecule.

The term “PEGylated” refers to conjugation with polyethylene glycol (PEG), which has been widely used as a drug carrier, given its high degree of biocompatibility and ease of modification. PEG can be coupled (e.g., covalently linked) to active agents through the hydroxy groups at the end of the PEG chain via chemical methods; however, PEG itself is limited to at most two active agents per molecule. In a different approach, copolymers of PEG and amino acids have been explored as novel biomaterial that would retain the biocompatibility of PEG, but that would have the added advantage of numerous attachment points per molecule (thus providing greater drug loading), and that can be synthetically designed to suit a variety of applications.

The term “gene” refers to a DNA sequence that comprises control and coding sequences necessary for the production of a polypeptide or precursor thereof. The polypeptide can be encoded by a full-length coding sequence or by any portion of the coding sequence so as the desired enzymatic activity is retained.

The term “native” refers to the typical form of a gene, a gene product, or a characteristic of that gene or gene product when isolated from a naturally occurring source. A native form is that which is most frequently observed in a natural population and is thus arbitrarily designated the normal or wild-type form. In contrast, the term “engineered,” “modified,” “variant,” or “mutant” refers to a gene or gene product that displays modification in sequence and functional properties (i.e., altered characteristics) when compared to the native gene or gene product.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for an RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

The term “therapeutically effective amount” as used herein refers to an amount of a therapeutic composition (such as a therapeutic polynucleotide and/or therapeutic polypeptide) that is employed in methods to achieve a therapeutic effect. The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

The term “K_{M}” as used herein refers to the Michaelis-Menten constant for an enzyme and is defined as the concentration of the specific substrate at which a given enzyme yields one-half its maximum velocity in an enzyme catalyzed reaction. The term “k_{cat}” as used herein refers to the turnover number or the number of substrate molecules each enzyme site converts to product per unit time, and in which the enzyme is working at maximum efficiency. The term “k_{cat}/K_{M}” as used herein is the specificity constant, which is a measure of how efficiently an enzyme converts a substrate into product.

The term “L-asparaginase” (L-ASP) refers to any enzyme that catalyzes the hydrolysis of asparagine to aspartic acid. For example, it includes bacterial forms of L-ASP, or particularly, E. coli forms of L-ASP.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of a L-ASP.

“Subject” and “patient” refer to either a human or non-human, such as primates, mammals, and vertebrates. In particular embodiments, the subject is a human.

### II. L-ASPARAGINASE

The enzyme-drug L-ASP has been used successfully for over 40 years to treat acute lymphoblastic leukemia (ALL). However, toxicity is a problem. Patients often cannot tolerate the 25-week course of L-ASP therapy that is frequently necessary to induce remission (Silverman et al., 2001). The toxicity has been attributed to L-ASP's glutaminase activity (Warrell et al., 1982; Kafkewitz and Bendich, 1983), but to complicate matters, the anticancer activity has also been attributed to glutaminase activity (Distasio et al., 1982; Ehsanipour et al., 2013; Fumarola et al., 2001; Durden et al., 1983; Distasio et al., 1976; Kitoh et al., 1992; Wu et al., 1978; Offman et al., 2011).

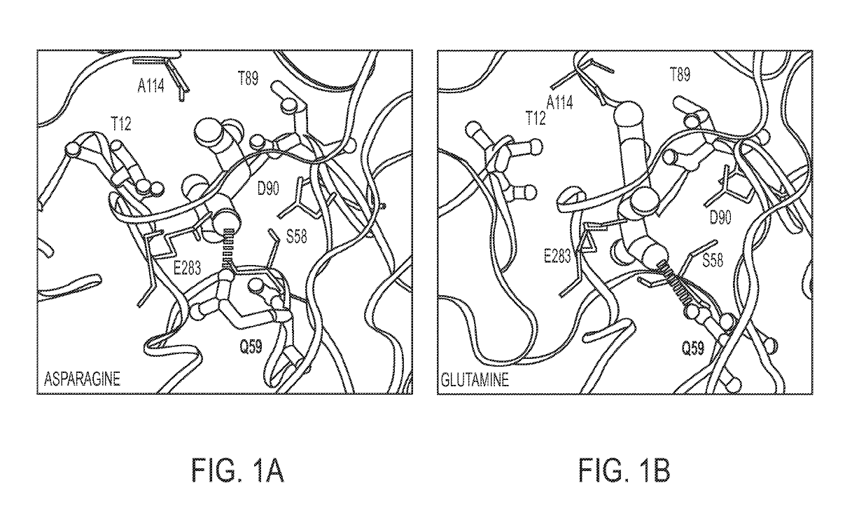

The present studies generated glutaminase-deficient derivatives of the clinically used E. coli L-ASP. The molecular structure of the E. coli asparaginase active site in complex with aspartic acid was first revealed in the 3ECA X-ray crystal structure (Swain et al., 1993) and later in a higher resolution structure, 1NNS (Sanches et al., 2003). Those structures revealed contacts between S58, Q59, D90, E283, and backbone groups of aspartic acid. N246 and N248 did not contact either substrate directly, despite the possibility that N248 might stabilize E283 in proximity of the ligand. Similar contacts were observed in the present molecular dynamics (MD) simulations, which identified Q59 as a catalytically nonessential residue with the greatest difference in contact pattern between the asparagine and glutamine substrates (Table 1 and FIG. 1).

In seeking a mutagenesis target that would suppress glutaminase activity but not asparaginase activity, a residue with the following four characteristics was desired: 1) does not participate directly in catalysis, 2) does not have a net charge, 3) contacts only the backbone of the substrate, and 4) preferentially contacts glutamine over asparagine. N248 was eliminated due to a lack of direct contact with the substrate and E283 for having charge. S58 and D90 satisfied 3 of the 4 criteria but contacted the two substrates with about equal frequency. Q59 satisfied all four criteria.

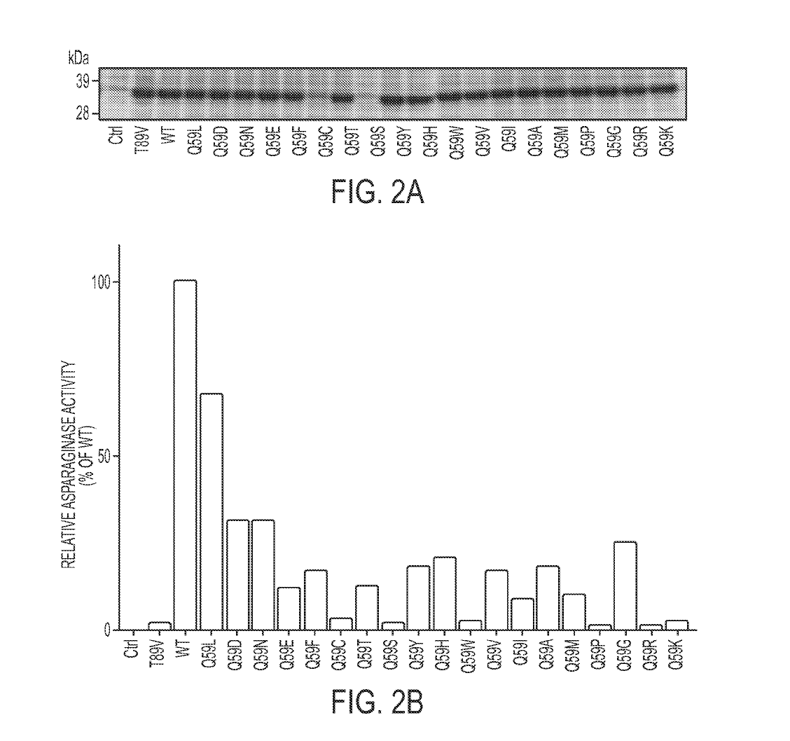

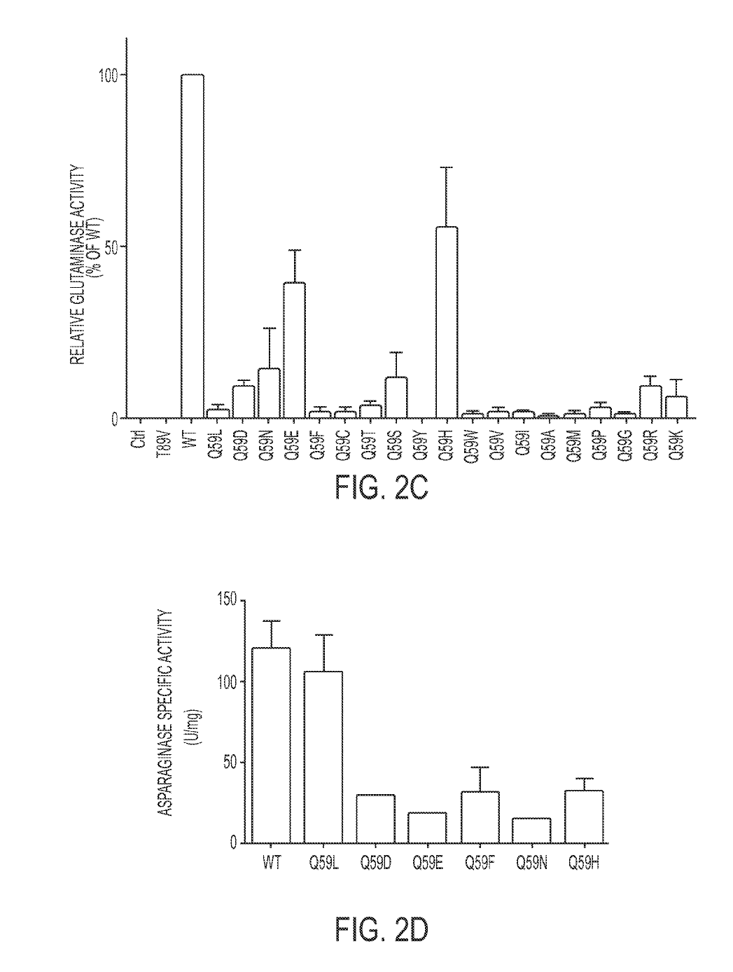

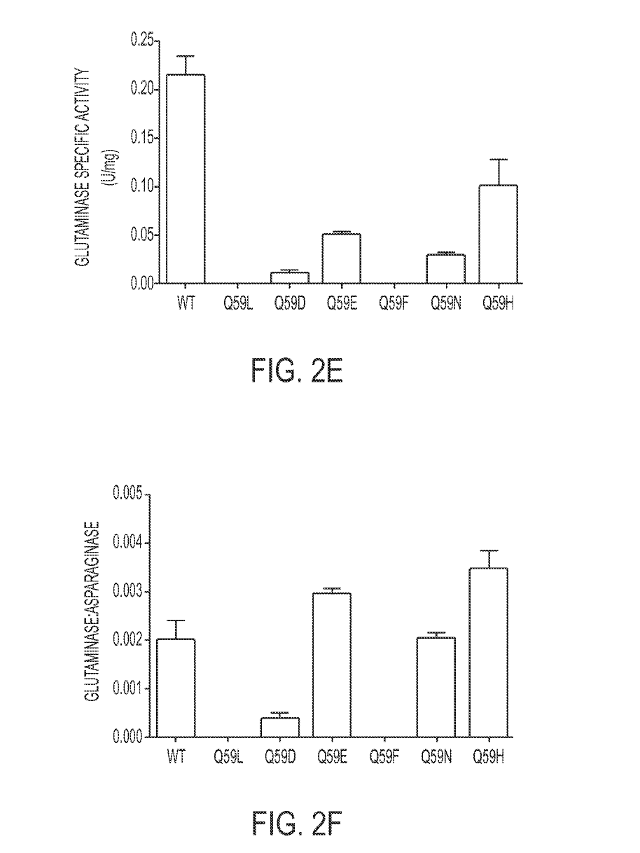

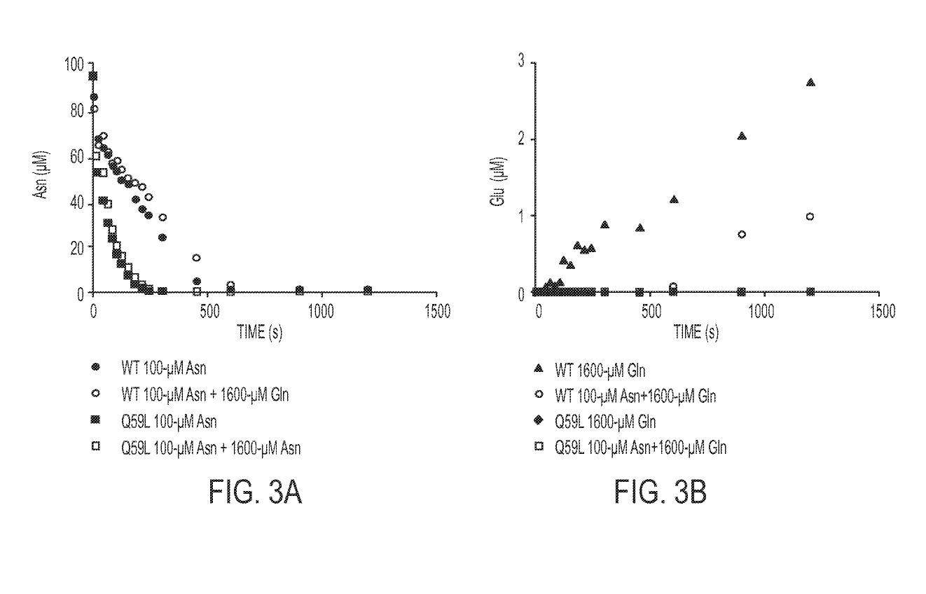

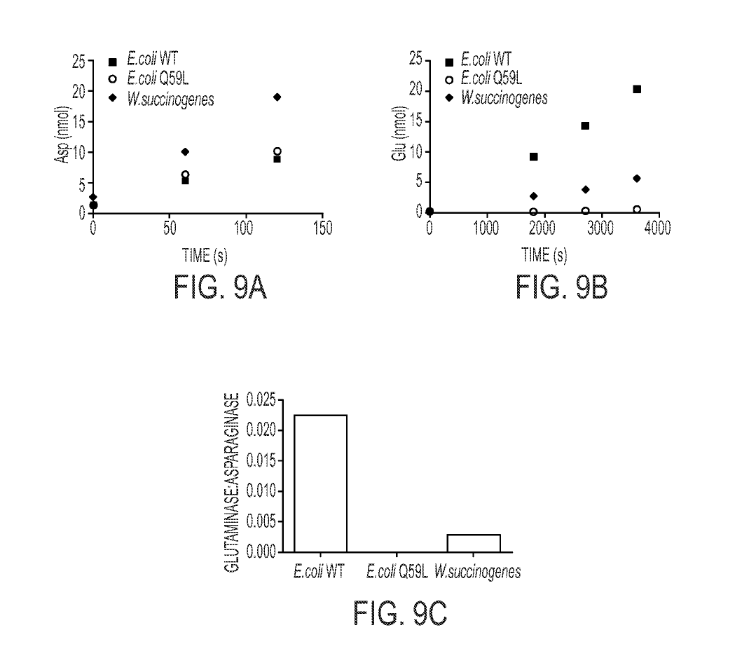

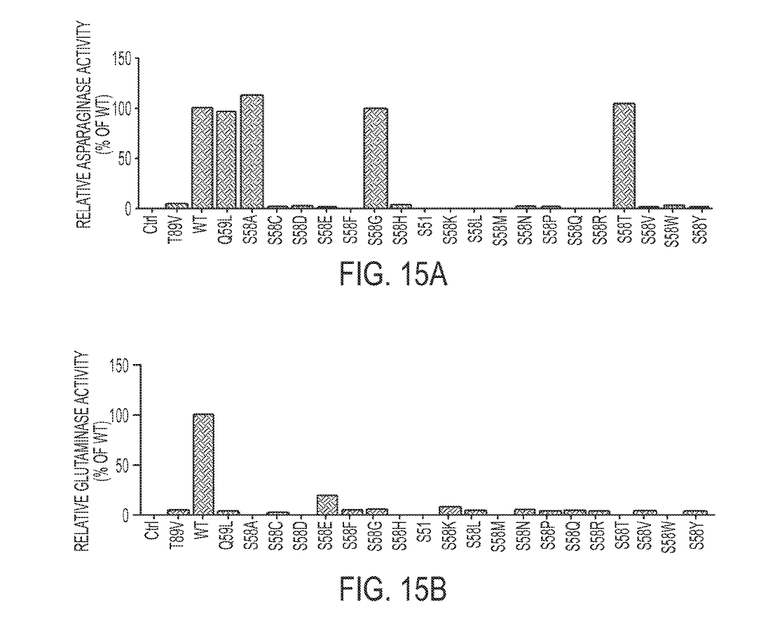

Having identified Q59 as the lead target, saturation mutagenesis was performed at position Q59 and a rapid colorimetric screening procedure was developed to measure the asparaginase and glutaminase activities of the resulting mutants. Importantly, the experimental screening results corroborated the in silico predictions: mutation of Q59 generally decreased glutaminase activity to a greater extent than asparaginase activity (FIG. 2). Of note, prior experimental mutagenesis at residue Q59 yielded three mutants (Q59A, Q59E, and Q59G) that exhibited dramatically reduced enzyme activity (Derst et al., 2000). Some of the Q59 mutants were further characterized using a sensitive LC-MS/MS assay. As a demonstration of the method's sensitivity, W. succinogenes L-ASP glutaminase activity was measured at 1.5×10⁻⁵ nmol/s (a level previously undetected by other methods) in the presence of 160 nM enzyme. Even with that sensitivity, however, Q59L showed no detectable glutaminase activity (FIG. 9C). In addition, its affinity for glutamine was sufficiently low that even high concentrations of glutamine (up to 16 mM) did not inhibit its asparaginase activity (FIG. 3A). In addition, saturation mutagenesis was performed at position S58 and it was found that S58G, S58T, and S58A resulted in decreased gluatminase activity but not asparaginase activity (FIGS. 15A-B).

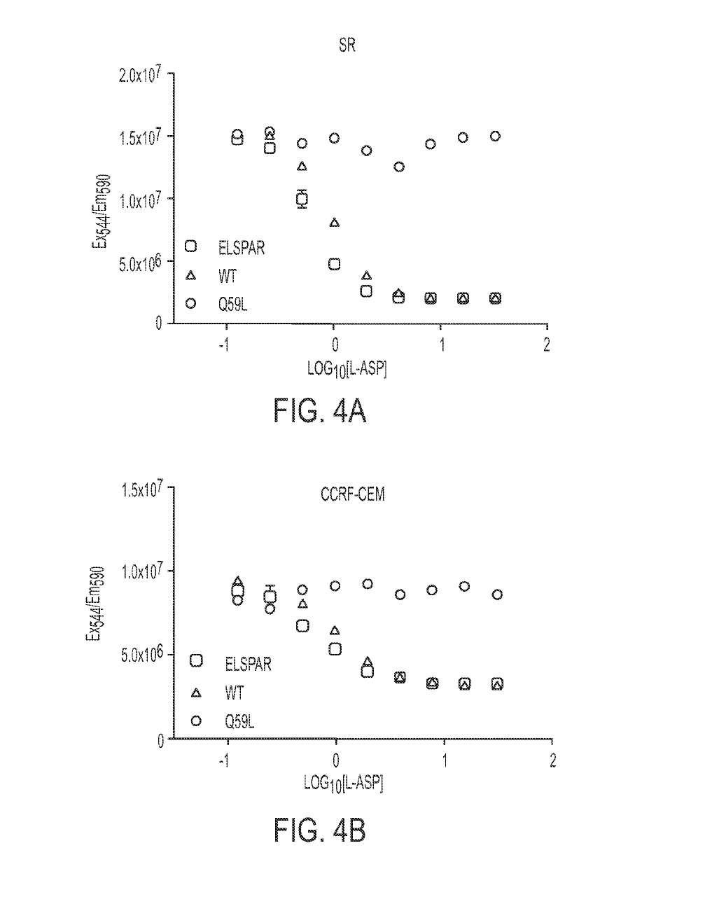

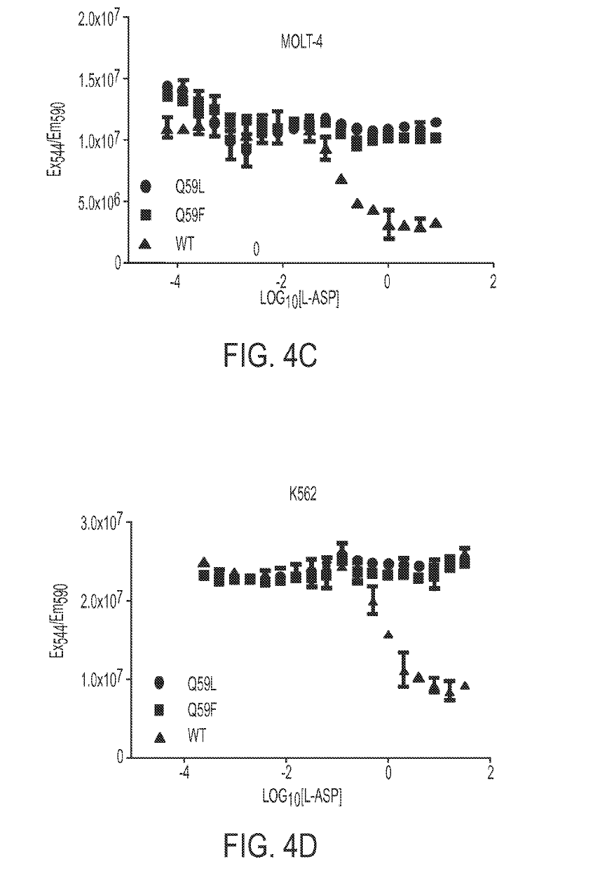

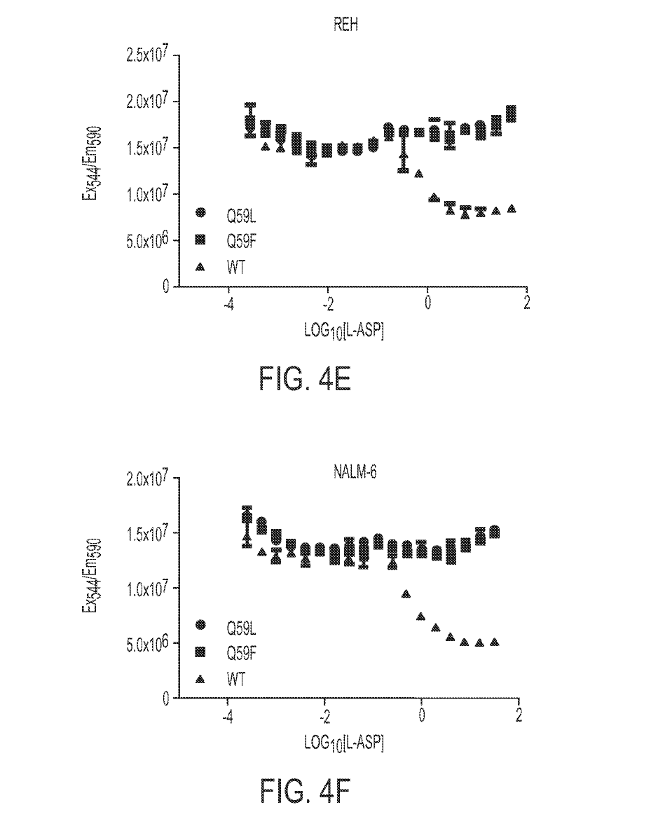

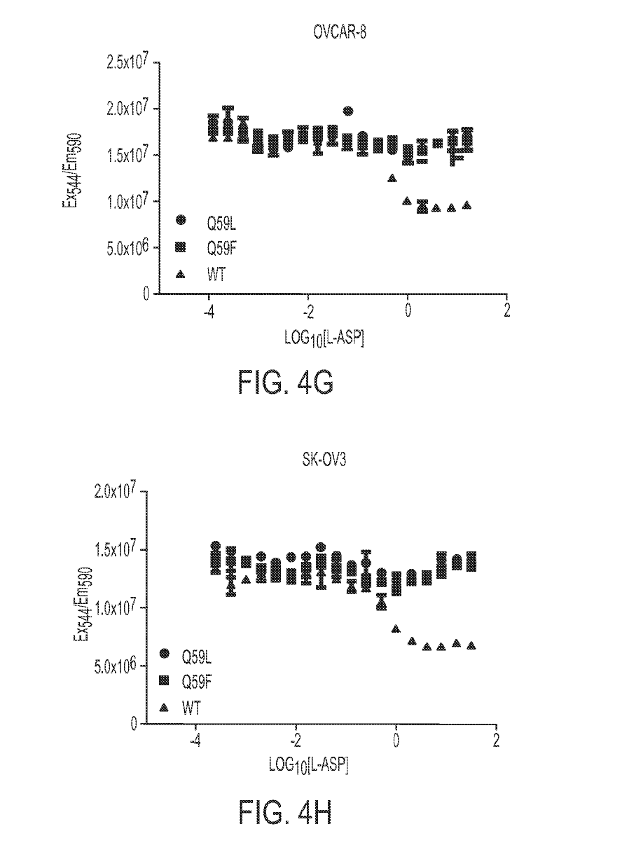

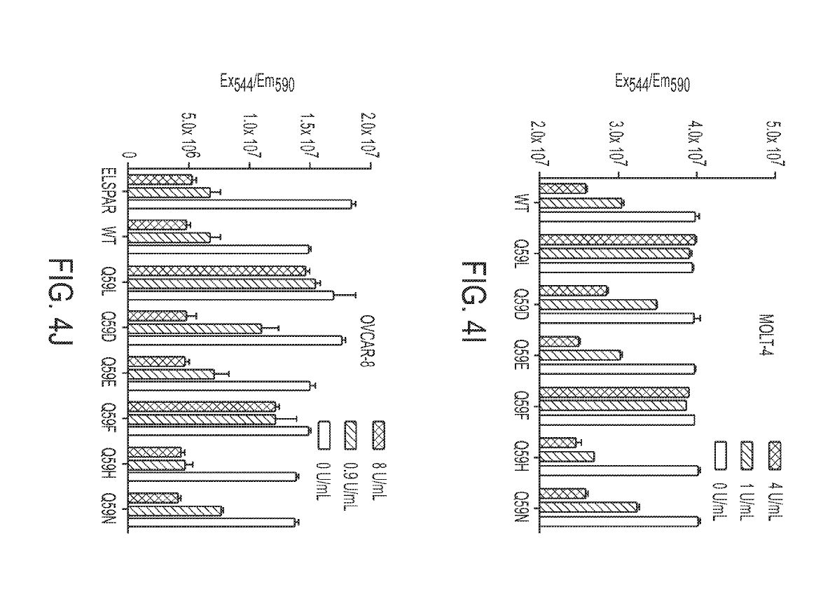

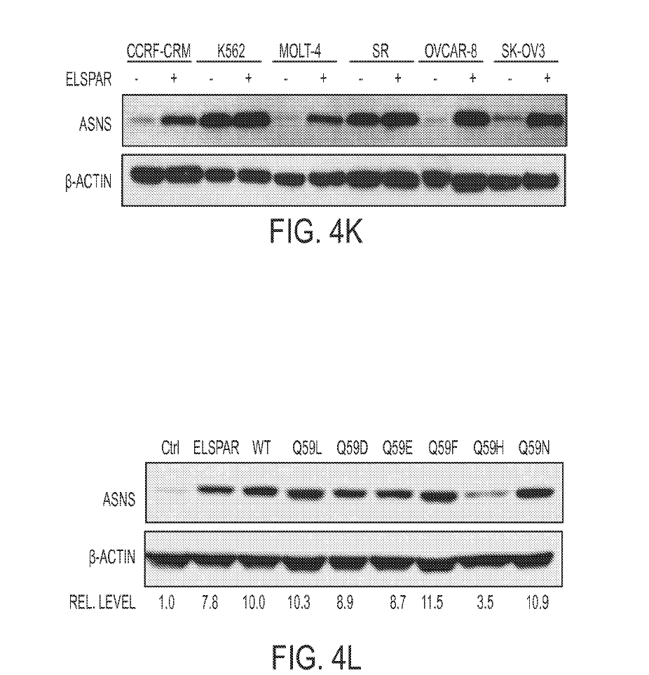

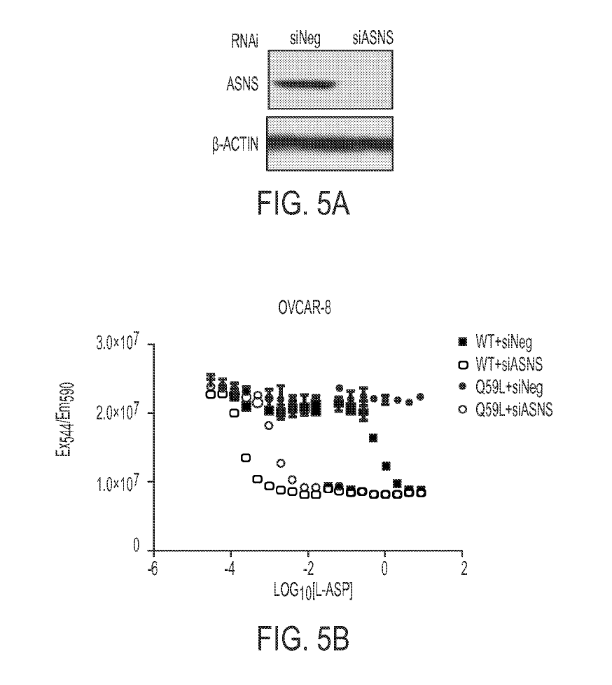

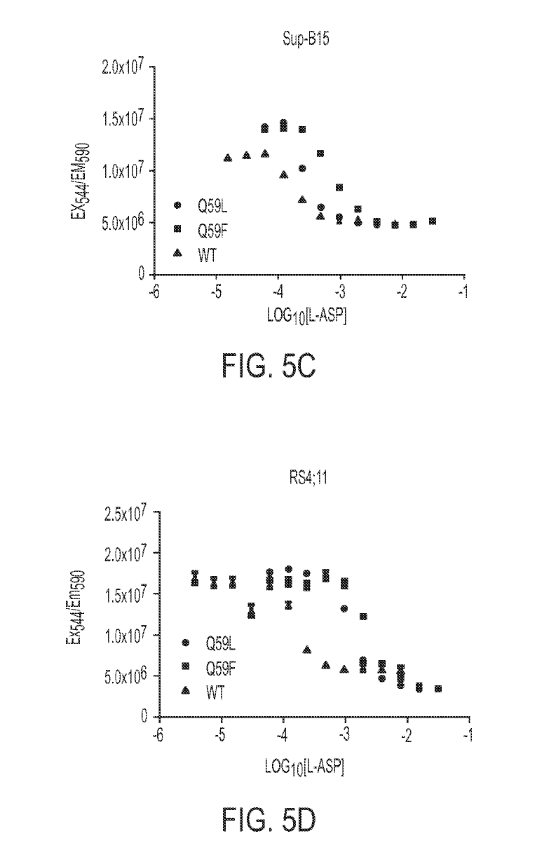

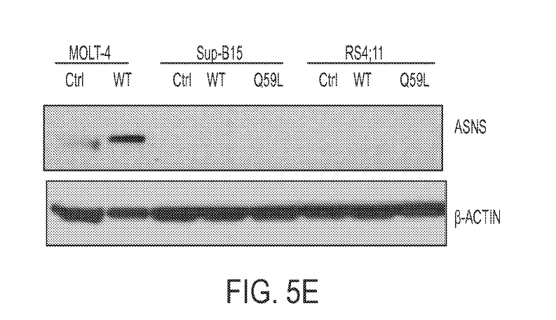

Because it has been demonstrated that ASNS expression is correlated with resistance to L-ASP (Bussey et al., 2006; Lorenzi et al., 2008; Scherf et al., 2000; Lorenzi et al., 2006; Aslanian et al., 2001; Haskell and Canellos, 1969; Horowitz et al., 1968; Hutson et al., 1997; Fine et al., 2005; Su et al., 2008; Leslie et al., 2006), the anticancer activity of WT, Q59L, and Q59F L-ASP was tested against six leukemia lines and two ovarian cancer lines that express ASNS. The glutaminase-deficient Q59L and Q59F L-ASP variants showed anticancer activity against ASNS-negative cell types (Sup-B15, RS4;11, and ASNS siRNA-treated OVCAR-8) (FIG. 5) but not ASNS-positive cell types (FIG. 4), suggesting that the glutaminase activity of L-ASP is not always required for anticancer activity. Notably, “ASNS-positive” cell types included lines such as MOLT-4, for which baseline ASNS expression was almost undetectable yet was induced following L-ASP treatment, and lines such as K562, for which baseline ASNS expression was high (FIG. 4K).

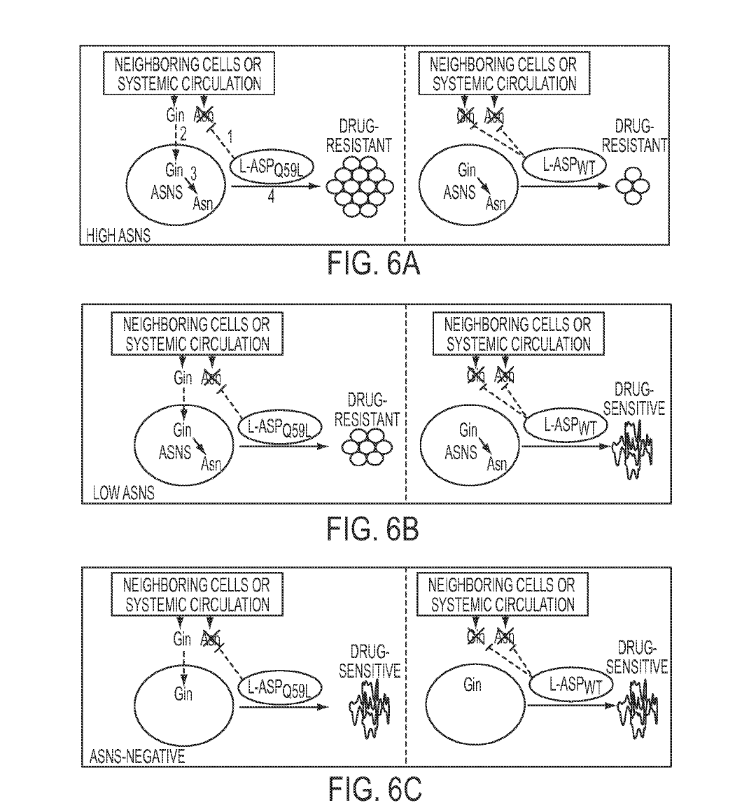

As illustrated in FIG. 6, cancer cells can be stratified into ASNS-negative and ASNS-positive, and the latter group can be further stratified into low and high ASNS. Only ASNS-positive cancer cells are able to use Gln imported from the extracellular environment to synthesize Asn, enabling them to proliferate regardless of the availability of extracellular Asn. Because Q59L L-ASP effectively depletes only Asn but not Gln, ASNS-positive cells are resistant to Q59L treatment (left panels, FIGS. 6A-B). In contrast, extracellular Asn is essential for proliferation of ASNS-negative cell types because of the inability to synthesize Asn endogenously (left panel, FIG. 6C). WT L-ASP, however, depletes the extracellular supply of both Asn and Gln due to its added glutaminase activity. High-ASNS cancer cells may continue to proliferate following such treatment if intracellular synthesis of Asn and Gln are sufficient (right panel, FIG. 6A). Low-ASNS cell types have reduced capacity to withstand such treatment; the reduced ability to produce Asn results in decreased cancer cell proliferation (right panel, FIG. 6B). ASNS-negative cell types cannot withstand WT L-ASP treatment, and, importantly, L-ASP glutaminase activity appears to be unnecessary for inhibiting proliferation of ASNS-negative cells (right panel, FIG. 6C), which are hypersensitive to asparaginase treatment without glutaminase activity.

The glutaminase activity of L-ASP has been implicated in many ALL treatment-associated side effects including immune suppression, pancreatitis, liver damage, and neurotoxicity (Kafkewitz and Bendich, 1983; Ollenschlager et al., 1988; Jenkins and Perlin, 1987; Villa et al., 1986; Reinert et al., 2006; Durden and Distasio, 1981). A potential strength of glutaminase-deficient L-ASP variants is, therefore, the possibility of improved therapeutic index if the modified L-ASP remains active against the cancer cells. For example, glutaminase-free L-ASP variants, such as Q59L, might not induce pancreatitis, as supported by the report that glutamine supplementation is highly effective in treating pancreatitis (Asrani et al., 2013).

In conclusion, the glutaminase activity of L-ASP is necessary for anticancer activity against cancer cells that express significant ASNS. However, ASNS-negative cancer cells are highly sensitive to asparaginase activity alone. Because the glutaminase activity of L-ASP is believed to be responsible for its toxicity, these findings suggest that a glutaminase-deficient L-ASP variant (e.g., Q59L, S58A, etc.) will exhibit greater therapeutic index than that of WT L-ASP against ASNS-negative cancers.

### III. L-ASP ENGINEERING

Some embodiments concern modified proteins and polypeptides. Particular embodiments concern a modified protein or polypeptide that exhibits at least one functional activity that is comparable to the unmodified version, preferably, the asparagine degrading activity. In further aspects, a modified protein or polypeptide may also exhibit a decrease in at least one functional activity relative to the unmodified version, preferably, the glutamine degrading activity. In still further aspects, the protein or polypeptide may be further modified to increase serum stability. Thus, when the present application refers to the function or activity of “modified protein” or a “modified polypeptide,” one of ordinary skill in the art would understand that this includes, for example, a protein or polypeptide that possesses an additional advantage over the unmodified protein or polypeptide, such as a decrease in glutamine degrading activity. In certain embodiments, the unmodified protein or polypeptide is a native L-ASP, preferably an E. coli L-ASP. It is specifically contemplated that embodiments concerning a “modified protein” may be implemented with respect to a “modified polypeptide,” and vice versa.

Determination of activity may be achieved using assays familiar to those of skill in the art, particularly with respect to the protein's activity, and may include for comparison purposes, for example, the use of native and/or recombinant versions of either the modified or unmodified protein or polypeptide. For example, the glutamine degrading activity may be determined by any assay to detect the production of any substrates resulting from the degradation of glutamine, such as the detection of glutamate.

In certain embodiments, a modified polypeptide, such as a modified L-ASP, may be identified based on its decrease in glutamine degrading activity. For example, substrate recognition sites of the unmodified polypeptide may be identified. This identification may be based on structural analysis or homology analysis. A population of mutants involving modifications of such substrate recognitions sites may be generated. In a further embodiment, mutants with decreased glutamine degrading activity may be selected from the mutant population. Selection of desired mutants may include methods for the detection of byproducts or products from glutamine degradation.

Modified proteins may possess deletions and/or substitutions of amino acids; thus, a protein with a deletion, a protein with a substitution, and a protein with a deletion and a substitution are modified proteins. In some embodiments, these modified proteins may further include insertions or added amino acids, such as with fusion proteins or proteins with linkers, for example. A “modified deleted protein” lacks one or more residues of the native protein, but may possess the specificity and/or activity of the native protein. A “modified deleted protein” may also have reduced immunogenicity or antigenicity. An example of a modified deleted protein is one that has an amino acid residue deleted from at least one antigenic region that is, a region of the protein determined to be antigenic in a particular organism, such as the type of organism that may be administered the modified protein.

Substitution or replacement variants typically contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

In addition to a deletion or substitution, a modified protein may possess an insertion of residues, which typically involves the addition of at least one residue in the polypeptide. This may include the insertion of a targeting peptide or polypeptide or simply a single residue. Terminal additions, called fusion proteins, are discussed below.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a control polypeptide are included, provided the biological activity of the protein is maintained. A modified protein may be biologically functionally equivalent to its native counterpart in certain aspects.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

### IV. ENZYMATIC ASPARAGINE DEGRADATION FOR THERAPY

In certain aspects, the polypeptides may be used for the treatment of diseases, including cancers that are sensitive to asparagine depletion with novel enzymes that deplete asparagine. The invention specifically discloses treatment methods using modified L-ASP with reduced glutamine degrading activity. Certain embodiments of the present invention provide novel asparaginase enzymes with reduced glutamine degrading activity for increased therapeutic efficacy.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

The engineered bacterial asparaginase derived from L-ASP may be used herein as an antitumor agent in a variety of modalities for depleting asparagine from a tumor cell, tumor tissue, or the circulation of a mammal with cancer, or for depletion of asparagine where its depletion is considered desirable.

Depletion can be conducted in vivo in the circulation of a mammal, in vitro in cases where asparagine depletion in tissue culture or other biological mediums is desired, and in ex vivo procedures where biological fluids, cells, or tissues are manipulated outside the body and subsequently returned to the body of the mammal. Depletion of asparagine from circulation, culture media, biological fluids, or cells is conducted to reduce the amount of asparagine accessible to the material being treated, and therefore comprises contacting the material to be depleted with an asparagine-degrading amount of the engineered bacterial asparaginase under asparagine-degrading conditions as to degrade the ambient asparagine in the material being contacted.

Because tumor cells may be dependent upon their nutrient medium for asparagine, the depletion may be directed to the nutrient source for the cells, and not necessarily the cells themselves. Therefore, in an in vivo application, treating a tumor cell includes contacting the nutrient medium for a population of tumor cells with the engineered L-ASP. In this embodiment, the medium may be blood, lymphatic fluid, spinal fluid and the like bodily fluid where asparagine depletion is desired.

Asparagine-degrading efficiency can vary widely depending upon the application, and typically depends upon the amount of asparagine present in the material, the desired rate of depletion, and the tolerance of the material for exposure to L-ASP. Asparagine levels in a material, and therefore rates of asparagine depletion from the material, can readily be monitored by a variety of chemical and biochemical methods well known in the art. Exemplary asparagine-degrading amounts are described further herein, and can range from 0.001 to 100 units (U) of engineered L-ASP, preferably about 0.01 to 10 U, and more preferably about 0.1 to 5 U engineered L-ASP per milliliter (mL) of material to be treated.

Asparagine-degrading conditions are buffer and temperature conditions compatible with the biological activity of an L-ASP enzyme, and include moderate temperature, salt, and pH conditions compatible with the enzyme, for example, physiological conditions. Exemplary conditions include about 4-40° C., ionic strength equivalent to about 0.05 to 0.2 M NaCl, and a pH of about 5 to 9, while physiological conditions are included.

In a particular embodiment, the invention contemplates methods of using engineered asparaginase as an antitumor agent, and therefore comprises contacting a population of tumor cells with a therapeutically effective amount of engineered asparaginase for a time period sufficient to inhibit tumor cell growth.

In one embodiment, the contacting in vivo is accomplished by administering, by intravenous or intraperitoneal injection, a therapeutically effective amount of a physiologically tolerable composition comprising an engineered L-ASP of this invention to a patient, thereby depleting the circulating asparagine source of the tumor cells present in the patient. The contacting of engineered L-ASP can also be accomplished by administering the engineered L-ASP into the tissue containing the tumor cells.

A therapeutically effective amount of an engineered L-ASP is a predetermined amount calculated to achieve the desired effect, i.e., to deplete asparagine in the tumor tissue or in a patient's circulation, and thereby cause the tumor cells to stop dividing. Thus, the dosage ranges for the administration of engineered L-ASP of the invention are those large enough to produce the desired effect in which the symptoms of tumor cell division and cell cycling are reduced. The dosage should not be so large as to cause adverse side effects, such as hyperviscosity syndromes, pulmonary edema, congestive heart failure, and the like. Generally, the dosage will vary with age of, condition of, sex of, and extent of the disease in the patient and can be determined by one of skill in the art. The dosage can be adjusted by the individual physician in the event of any complication.

For example, a therapeutically effective amount of an engineered L-ASP may be an amount such that when administered in a physiologically tolerable composition is sufficient to achieve a intravascular (plasma) or local concentration of from about 0.001 to about 100 units (U) per mL, preferably above about 0.1 U, and more preferably above 1 U engineered L-ASP per mL. Typical dosages can be administered based on body weight, and are in the range of about 5-1000 U/kilogram (kg)/day, preferably about 5-100 U/kg/day, more preferably about 10-50 U/kg/day, and more preferably about 20-40 U/kg/day.

The engineered L-ASP can be administered parenterally by injection or by gradual infusion over time. The engineered L-ASP can be administered intravenously, intraperitoneally, orally, intramuscularly, subcutaneously, intracavity, transdermally, dermally, can be delivered by peristaltic means, can be injected directly into the tissue containing the tumor cells, or can be administered by a pump connected to a catheter that may contain a potential biosensor for asparagine.

The therapeutic compositions containing engineered L-ASP are conventionally administered intravenously, as by injection of a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required diluent, i.e., carrier, or vehicle.

The compositions are administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for initial administration and booster shots are also contemplated and are typified by an initial administration followed by repeated doses at one or more hour intervals by a subsequent injection or other administration. Exemplary multiple administrations are described herein and are particularly preferred to maintain continuously high serum and tissue levels of engineered L-ASP and conversely low serum and tissue levels of asparagine. Alternatively, continuous intravenous infusion sufficient to maintain concentrations in the blood in the ranges specified for in vivo therapies are contemplated.

### V. CONJUGATES

Compositions and methods of the present invention involve engineered L-ASP, such as by forming conjugates with heterologous peptide segments or polymers, such as polyethylene glycol. In further aspects, the engineered L-ASP may be linked to PEG to increase the hydrodynamic radius of the enzyme and hence increase the serum persistence. In certain aspects, the disclosed polypeptide may be conjugated to any targeting agent, such as a ligand having the ability to specifically and stably bind to an external receptor or binding site on a tumor cell (U.S. Patent Publ. 2009/0304666).

A. Fusion Proteins

Certain embodiments of the present invention concern fusion proteins. These molecules may have the modified L-ASP linked at the N- or C-terminus to a heterologous domain. For example, fusions may also employ leader sequences from other species to permit the recombinant expression of a protein in a heterologous host. Another useful fusion includes the addition of a protein affinity tag, such as a serum albumin affinity tag or six histidine residues, or an immunologically active domain, such as an antibody epitope, preferably cleavable, to facilitate purification of the fusion protein. Non-limiting affinity tags include polyhistidine, chitin binding protein (CBP), maltose binding protein (MBP), and glutathione-S-transferase (GST).

In a particular embodiment, the L-ASP may be linked to a peptide that increases the in vivo half-life, such as an XTEN polypeptide (Schellenberger et al., 2009), IgG Fc domain, albumin, or albumin binding peptide.

Methods of generating fusion proteins are well known to those of skill in the art. Such proteins can be produced, for example, by de novo synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that is spliced between the polypeptides connected in tandem. The linker would be of sufficient length to allow proper folding of the resulting fusion protein.

B. Linkers

In certain embodiments, the engineered L-ASP may be chemically conjugated using bifunctional cross-linking reagents or fused at the protein level with peptide linkers.

Bifunctional cross-linking reagents have been extensively used for a variety of purposes, including preparation of affinity matrices, modification and stabilization of diverse structures, identification of ligand and receptor binding sites, and structural studies. Suitable peptide linkers may also be used to link the engineered L-ASP, such as Gly-Ser linkers.

Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino-, sulfhydryl-, guanidine-, indole-, carboxyl-specific groups. Of these, reagents directed to free amino groups have become especially popular because of their commercial availability, ease of synthesis, and the mild reaction conditions under which they can be applied.

A majority of heterobifunctional cross-linking reagents contain a primary amine-reactive group and a thiol-reactive group. In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling, in one example, of aldehydes to free thiols. The cross-linking reagent can be modified to cross-link various functional groups.

Additionally, any other linking/coupling agents and/or mechanisms known to those of skill in the art may be used to combine engineered L-ASP, such as, for example, antibody-antigen interaction, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo. These linkers are thus one group of linking agents.

In addition to hindered cross-linkers, non-hindered linkers also can be employed in accordance herewith. Other useful cross-linkers, not considered to contain or generate a protected disulfide, include SATA, SPDP, and 2-iminothiolane (Wawrzynczak and Thorpe, 1987). The use of such cross-linkers is well understood in the art. Another embodiment involves the use of flexible linkers.

Once chemically conjugated, the peptide generally will be purified to separate the conjugate from unconjugated agents and from other contaminants. A large number of purification techniques are available for use in providing conjugates of a sufficient degree of purity to render them clinically useful.

Purification methods based upon size separation, such as gel filtration, gel permeation, or high performance liquid chromatography, will generally be of most use. Other chromatographic techniques, such as Blue-Sepharose separation, may also be used. Conventional methods to purify the fusion proteins from inclusion bodies may be useful, such as using weak detergents, such as sodium N-lauroyl-sarcosine (SLS).

C. PEGylation

In certain aspects of the invention, methods and compositions related to PEGylation of engineered L-ASP are disclosed. For example, the engineered L-ASP may be PEGylated in accordance with the methods disclosed herein.

PEGylation is the process of covalent attachment of poly(ethylene glycol) polymer chains to another molecule, normally a drug or therapeutic protein. PEGylation is routinely achieved by incubation of a reactive derivative of PEG with the target macromolecule. The covalent attachment of PEG to a drug or therapeutic protein can “mask” the agent from the host's immune system (reduced immunogenicity and antigenicity) or increase the hydrodynamic size (size in solution) of the agent, which prolongs its circulatory time by reducing renal clearance. PEGylation can also provide water solubility to hydrophobic drugs and proteins.

The first step of the PEGylation is the suitable functionalization of the PEG polymer at one or both terminals. PEGs that are activated at each terminus with the same reactive moiety are known as “homobifunctional,” whereas if the functional groups present are different, then the PEG derivative is referred as “heterobifunctional” or “heterofunctional.” The chemically active or activated derivatives of the PEG polymer are prepared to attach the PEG to the desired molecule.

The choice of the suitable functional group for the PEG derivative is based on the type of available reactive group on the molecule that will be coupled to the PEG. For proteins, typical reactive amino acids include lysine, cysteine, histidine, arginine, aspartic acid, glutamic acid, serine, threonine, and tyrosine. The N-terminal amino group and the C-terminal carboxylic acid can also be used.

The techniques used to form first generation PEG derivatives are generally reacting the PEG polymer with a group that is reactive with hydroxyl groups, typically anhydrides, acid chlorides, chloroformates, and carbonates. In the second generation PEGylation chemistry more efficient functional groups, such as aldehyde, esters, amides, etc., are made available for conjugation.

As applications of PEGylation have become more and more advanced and sophisticated, there has been an increase in need for heterobifunctional PEGs for conjugation. These heterobifunctional PEGs are very useful in linking two entities, where a hydrophilic, flexible, and biocompatible spacer is needed. Preferred end groups for heterobifunctional PEGs are maleimide, vinyl sulfones, pyridyl disulfide, amine, carboxylic acids, and NHS esters.

The most common modification agents, or linkers, are based on methoxy PEG (mPEG) molecules. Their activity depends on adding a protein-modifying group to the alcohol end. In some instances polyethylene glycol (PEG diol) is used as the precursor molecule. The diol is subsequently modified at both ends in order to make a hetero- or homo-dimeric PEG-linked molecule.

Proteins are generally PEGylated at nucleophilic sites, such as unprotonated thiols (cysteinyl residues) or amino groups. Examples of cysteinyl-specific modification reagents include PEG maleimide, PEG iodoacetate, PEG thiols, and PEG vinylsulfone. All four are strongly cysteinyl-specific under mild conditions and neutral to slightly alkaline pH but each has some drawbacks. The thioether formed with the maleimides can be somewhat unstable under alkaline conditions so there may be some limitation to formulation options with this linker. The carbamothioate linkage formed with iodo PEGs is more stable, but free iodine can modify tyrosine residues under some conditions. PEG thiols form disulfide bonds with protein thiols, but this linkage can also be unstable under alkaline conditions. PEG-vinylsulfone reactivity is relatively slow compared to maleimide and iodo PEG; however, the thioether linkage formed is quite stable. Its slower reaction rate also can make the PEG-vinylsulfone reaction easier to control.

Site-specific PEGylation at native cysteinyl residues is seldom carried out, since these residues are usually in the form of disulfide bonds or are required for biological activity. On the other hand, site-directed mutagenesis can be used to incorporate cysteinyl PEGylation sites for thiol-specific linkers. The cysteine mutation must be designed such that it is accessible to the PEGylation reagent and is still biologically active after PEGylation.

Amine-specific modification agents include PEG NHS ester, PEG tresylate, PEG aldehyde, PEG isothiocyanate, and several others. All react under mild conditions and are very specific for amino groups. The PEG NHS ester is probably one of the more reactive agents; however, its high reactivity can make the PEGylation reaction difficult to control on a large scale. PEG aldehyde forms an imine with the amino group, which is then reduced to a secondary amine with sodium cyanoborohydride. Unlike sodium borohydride, sodium cyanoborohydride will not reduce disulfide bonds. However, this chemical is highly toxic and must be handled cautiously, particularly at lower pH where it becomes volatile.

Due to the multiple lysine residues on most proteins, site-specific PEGylation can be a challenge. Fortunately, because these reagents react with unprotonated amino groups, it is possible to direct the PEGylation to lower-pK amino groups by performing the reaction at a lower pH. Generally the pK of the alpha-amino group is 1-2 pH units lower than the epsilon-amino group of lysine residues. By PEGylating the molecule at pH 7 or below, high selectivity for the N-terminus frequently can be attained. However, this is only feasible if the N-terminal portion of the protein is not required for biological activity. Still, the pharmacokinetic benefits from PEGylation frequently outweigh a significant loss of in vitro bioactivity, resulting in a product with much greater in vivo bioactivity regardless of PEGylation chemistry.

There are several parameters to consider when developing a PEGylation procedure. Fortunately, there are usually no more than four or five key parameters. The “design of experiments” approach to optimization of PEGylation conditions can be very useful. For thiol-specific PEGylation reactions, parameters to consider include: protein concentration, PEG-to-protein ratio (on a molar basis), temperature, pH, reaction time, and in some instances, the exclusion of oxygen. (Oxygen can contribute to intermolecular disulfide formation by the protein, which will reduce the yield of the PEGylated product.) The same factors should be considered (with the exception of oxygen) for amine-specific modification except that pH may be even more critical, particularly when targeting the N-terminal amino group.

For both amine- and thiol-specific modifications, the reaction conditions may affect the stability of the protein. This may limit the temperature, protein concentration, and pH. In addition, the reactivity of the PEG linker should be known before starting the PEGylation reaction. For example, if the PEGylation agent is only 70 percent active, the amount of PEG used should ensure that only active PEG molecules are counted in the protein-to-PEG reaction stoichiometry.

D. Red Blood Cell Encapsulation

“Red Blood Cells” (RBCs), or erythrocytes, are terminally differentiated cells derived from hematopoietic stem cells. They lack a nucleus and most cellular organelles. RBCs contain hemoglobin to carry oxygen from the lungs to the peripheral tissues. They also carry CO₂ produced by cells during metabolism out of the tissues and back to the lungs for release during exhale. RBCs are produced in the bone marrow in response to blood hypoxia which is mediated by release of erythropoietin (EPO) by the kidney. EPO causes an increase in the number of proerythroblasts and shortens the time required for full RBC maturation. After approximately 120 days, since the RBC do not contain a nucleus or any other regenerative capabilities, the cells are removed from circulation by either the phagocytic activities of macrophages in the liver, spleen and lymph nodes (−90%) or by hemolysis in the plasma (˜10%). Following macrophage engulfment, chemical components of the RBC are broken down within vacuoles of the macrophages due to the action of lysosomal enzymes.

Red blood cells (RBCs), or erythrocytes, are the major cellular component of blood. In fact, RBCs account for one quarter of the cells in a human. In humans mature RBCs lack a nucleus and many other organelles, and are full of hemoglobin to facilitate their job of taking oxygen from the lungs and delivering it to the peripheral tissues. RBCs are developed in the bone marrow from CD34+ hematopoietic stem cells and have a half-life of approximately 100 to 120 days.

The use of red blood cells as carriers for transporting biologically active substances, encapsulated in the red blood cells or bound to their surface, and delivered to a target has been envisaged in several publications. These proteins can be produced and trapped within the RBC such that enzyme activity occurs following diffusion of a substrate into the RBC while it is traveling through the blood stream. Alternatively, the protein (e.g., enzyme) can be anchored to the surface of the RBC where it will act on the substrate outside of the RBC in the serum. The protein may also be released or secreted into the blood stream by the RBC. Various techniques have been developed to enable the encapsulation of proteins in erythrocytes (red blood cells). Accordingly, numerous devices have been designed to assist or simplify the encapsulation procedure. The encapsulation methods known in the art include osmotic pulse (swelling) and reconstitution of cells, controlled lysis and resealing, incorporation of liposomes, and electroporation.

Additionally, U.S. Pat. Nos. 4,192,869, 4,321,259, and 4,473,563 describe a method whereby fluid-charged lipid vesicles are fused with erythrocyte membranes, depositing their contents into the red blood cells. In this manner, it is possible to transport proteins into erythrocytes.

In accordance with the liposome technique, the protein is dissolved in a buffer until the solution is saturated and a mixture of lipid vesicles is suspended in the solution. The suspension is then subjected to ultrasonic treatment or an injection process, and then centrifuged. The upper suspension contains small lipid vesicles containing the protein, which are then collected. Erythrocytes are added to the collected suspension and incubated, during which time the lipid vesicles containing the protein fuse with the cell membranes of the erythrocytes, thereby depositing their contents into the interior of the erythrocyte. The modified erythrocytes are then washed and added to plasma to complete the product.

A method of lysing and the resealing red blood cells has also been developed (Nicolau et al., 1985; U.S. Pat. Nos. 4,752,856 and 4,652,449). The technique is best characterized as a continuous flow dialysis system which functions in a manner similar to the osmotic pulse technique. Specifically, the primary compartment of at least one dialysis element is continuously supplied with an aqueous suspension of erythrocytes while the secondary compartment of the dialysis element contains an aqueous solution which is hypotonic with respect to the erythrocyte suspension. The hypotonic solution causes the erythrocytes to lyse. The erythrocyte lysate is then contacted with the biologically active substance to be incorporated into the erythrocyte. To reseal the membranes of the erythrocytes, the osmotic and/or oncotic pressure of the erythrocyte lysate is increased and the suspension of resealed erythrocytes is recovered.

In U.S. Pat. Nos. 4,874,690 and 5,043,261 a related technique involving lyophilization and reconstitution of red blood cells is disclosed. As part of the process of reconstituting the red blood cells, the addition of various polyanions, including inositol hexaphosphate, is described. Treatment of the red blood cells according to the process disclosed results in a cell with unaffected activity. Presumably, the biologically active substance is incorporated into the cell during the reconstitution process, thereby maintaining the activity of the hemoglobin.

In U.S. Pat. Nos. 4,478,824 and 4,931,276, another method and apparatus is described for introducing biologically active substances into mammalian red blood cells by effectively lysing and resealing the cells. The procedure is described as the “osmotic pulse technique.” In practicing the osmotic pulse technique, a supply of packed red blood cells is suspended and incubated in a solution containing a compound which readily diffuses into and out of the cells, the concentration of the compound being sufficient to cause diffusion thereof into the cells so that the contents of the cells become hypertonic. Next, a transmembrane ionic gradient is created by diluting the solution containing the hypertonic cells with an essentially isotonic aqueous medium in the presence of at least on desired agent to be introduced, thereby causing diffusion of water into the cells with a consequent swelling and an increases in permeability of the outer membranes of the cells. This “osmotic pulse” causes the diffusion of water into the cells and a resultant swelling of the cells which increase the permeability of the outer cell membrane to the desired agent. The increase in permeability of the membrane is maintained for a period of time sufficient only to permit transport of least one agent into the cells and diffusion of the compound out of the cells. Polyanions that may be used in practicing the osmotic pulse technique include pyrophosphate, tripolyphosphate, phosphorylated inositols, 2,3-diphosphogly-cerate (DPG), adenosine triphosphate, heparin, and polycar-boxylic acids which are water-soluble, and non-disruptive to the lipid outer bilayer membranes of red blood cells.

Another method for encapsulating various biologically-active substances in erythrocytes is electroporation. Electroporation has been used for encapsulation of foreign molecules in different cell types including red blood cells (Mouneimne et al., 1990). The process of electroporation involves the formation of pores in the cell membranes, or in any vesicles, by the application of electric field pulses across a liquid cell suspension containing the cells or vesicles. During the poration process, cells are suspended in a liquid media and then subjected to an electric field pulse. The medium may be electrolyte, non-electrolyte, or a mixture of electrolytes and non-electrolytes. The strength of the electric field applied to the suspension and the length of the pulse (the time that the electric field is applied to a cell suspension) varies according to the cell type. To create a pore in a cell's outer membrane, the electric field must be applied for such a length of time and at such a voltage as to create a set potential across the cell membrane for a period of time long enough to create a pore.

### VI. PROTEINS AND PEPTIDES

In certain embodiments, the present invention concerns compositions comprising at least one protein or peptide, such as an engineered L-ASP. These peptides may be comprised in a fusion protein or conjugated to an agent as described supra.

As used herein, a protein or peptide generally refers, but is not limited to, a protein of greater than about 200 amino acids, up to a full-length sequence translated from a gene; a polypeptide of greater than about 100 amino acids; and/or a peptide of from about 3 to about 100 amino acids. For convenience, the terms “protein,” “polypeptide,” and “peptide” are used interchangeably herein.

As used herein, an “amino acid residue” refers to any naturally occurring amino acid, any amino acid derivative, or any amino acid mimic known in the art. In certain embodiments, the residues of the protein or peptide are sequential, without any non-amino acids interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid.

Proteins or peptides may be made by any technique known to those of skill in the art, including the expression of proteins, polypeptides, or peptides through standard molecular biological techniques, the isolation of proteins or peptides from natural sources, or the chemical synthesis of proteins or peptides. The nucleotide and protein, polypeptide, and peptide sequences corresponding to various genes have been previously disclosed, and may be found at computerized databases known to those of ordinary skill in the art. One such database is the National Center for Biotechnology Information's Genbank and GenPept databases (available on the world wide web at ncbi.nlm.nih gov/). The coding regions for known genes may be amplified and/or expressed using the techniques disclosed herein or as would be known to those of ordinary skill in the art. Alternatively, various commercial preparations of proteins, polypeptides, and peptides are known to those of skill in the art.

### VII. NUCLEIC ACIDS AND VECTORS

In certain aspects of the invention, nucleic acid sequences encoding an engineered L-ASP or a fusion protein containing a modified L-ASP may be disclosed. Depending on which expression system is used, nucleic acid sequences can be selected based on conventional methods. For example, an open reading frame encoding an engineered L-ASP may be codon optimized for expression in specific organisms. Various vectors may be also used to express the protein of interest, such as engineered L-ASP. Exemplary vectors include, but are not limited, plasmid vectors, viral vectors, transposon, or liposome-based vectors.

### VIII. HOST CELLS

Host cells may be any that may be transformed to allow the expression and secretion of engineered L-ASP and conjugates thereof. The host cells may be bacteria, mammalian cells, yeast, or filamentous fungi. Various bacteria include Escherichia and Bacillus. Yeasts belonging to the genera Saccharomyces, Kiuyveromyces, Hansenula, or Pichia would find use as an appropriate host cell. Various species of filamentous fungi may be used as expression hosts, including the following genera: Aspergillus, Trichoderma, Neurospora, Penicillium, Cephalosporium, Achlya, Podospora, Endothia, Mucor, Cochliobolus, and Pyricularia.

Examples of usable host organisms include bacteria, e.g., Escherichia coli MC1061, derivatives of Bacillus subtilis BRB1 (Sibakov et al., 1984), Staphylococcus aureus SAI123 (Lordanescu, 1975) or Streptococcus lividans (Hopwood et al., 1985); yeasts, e.g., Saccharomyces cerevisiae AH 22 (Mellor et al., 1983) or Schizosaccharomyces pombe; and filamentous fungi, e.g., Aspergillus nidulans, Aspergillus awamori (Ward, 1989), or Trichoderma reesei (Penttila et al., 1987; Harkki et al., 1989).

Examples of mammalian host cells include Chinese hamster ovary cells (CHO-K1; ATCC CCL61), rat pituitary cells (GH1; ATCC CCL82), HeLa S3 cells (ATCC CCL2.2), rat hepatoma cells (H-4-II-E; ATCCCRL 1548), SV40-transformed monkey kidney cells (COS-1; ATCC CRL 1650), and murine embryonic cells (NIH-3T3; ATCC CRL 1658). The foregoing being illustrative but not limitative of the many possible host organisms known in the art. In principle, all hosts capable of secretion can be used whether prokaryotic or eukaryotic.

Mammalian host cells expressing the engineered L-ASP and/or their fusion proteins are cultured under conditions typically employed to culture the parental cell line. Generally, cells are cultured in a standard medium containing physiological salts and nutrients, such as standard RPMI, MEM, IMEM, or DMEM, typically supplemented with 5%-10% serum, such as fetal bovine serum. Culture conditions are also standard, e.g., cultures are incubated at 37° C. in stationary or roller cultures until desired levels of the proteins are achieved.

### IX. PROTEIN PURIFICATION

Protein purification techniques are well known to those of skill in the art. These techniques involve, at one level, the homogenization and crude fractionation of the cells, tissue, or organ to polypeptide and non-polypeptide fractions. The protein or polypeptide of interest may be further purified using chromatographic and electrophoretic techniques to achieve partial or complete purification (or purification to homogeneity) unless otherwise specified. Analytical methods particularly suited to the preparation of a pure peptide are ion-exchange chromatography, gel exclusion chromatography, polyacrylamide gel electrophoresis, affinity chromatography, immunoaffinity chromatography, and isoelectric focusing. A particularly efficient method of purifying peptides is fast-performance liquid chromatography (FPLC) or even high-performance liquid chromatography (HPLC).

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various techniques suitable for use in protein purification are well known to those of skill in the art. These include, for example, precipitation with ammonium sulphate, PEG, antibodies and the like, or by heat denaturation, followed by centrifugation; chromatography steps, such as ion exchange, gel filtration, reverse phase, hydroxyapatite, and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

There is no general requirement that the protein or peptide will always be provided in its most purified state. Indeed, it is contemplated that less substantially purified products may have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

In certain embodiments a protein or peptide may be isolated or purified, for example, an engineered L-ASP, a fusion protein containing an engineered L-ASP, or an engineered L-ASP post PEGylation. For example, a His tag or an affinity epitope may be comprised in such an engineered L-ASP to facilitate purification. Affinity chromatography is a chromatographic procedure that relies on the specific affinity between a substance to be isolated and a molecule to which it can specifically bind. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that does not adsorb molecules to any significant extent and that has a broad range of chemical, physical, and thermal stability. The ligand should be coupled in such a way as to not affect its binding properties. The ligand should also provide relatively tight binding. It should be possible to elute the substance without destroying the sample or the ligand.

Size exclusion chromatography (SEC) is a chromatographic method in which molecules in solution are separated based on their size, or in more technical terms, their hydrodynamic volume. It is usually applied to large molecules or macromolecular complexes, such as proteins and industrial polymers. Typically, when an aqueous solution is used to transport the sample through the column, the technique is known as gel filtration chromatography, versus the name gel permeation chromatography, which is used when an organic solvent is used as a mobile phase.

The underlying principle of SEC is that particles of different sizes will elute (filter) through a stationary phase at different rates. This results in the separation of a solution of particles based on size. Provided that all the particles are loaded simultaneously or near simultaneously, particles of the same size should elute together. Each size exclusion column has a range of molecular weights that can be separated. The exclusion limit defines the molecular weight at the upper end of this range and is where molecules are too large to be trapped in the stationary phase. The permeation limit defines the molecular weight at the lower end of the range of separation and is where molecules of a small enough size can penetrate into the pores of the stationary phase completely and all molecules below this molecular mass are so small that they elute as a single band.

High-performance liquid chromatography (or high-pressure liquid chromatography, HPLC) is a form of column chromatography used frequently in biochemistry and analytical chemistry to separate, identify, and quantify compounds. HPLC utilizes a column that holds chromatographic packing material (stationary phase), a pump that moves the mobile phase(s) through the column, and a detector that shows the retention times of the molecules. Retention time varies depending on the interactions between the stationary phase, the molecules being analyzed, and the solvent(s) used.

### X. PHARMACEUTICAL COMPOSITIONS

It is contemplated that an L-ASP can be administered systemically or locally to inhibit tumor cell growth and, most preferably, to kill cancer cells in cancer patients with locally advanced or metastatic cancers. They can be administered intravenously, intrathecally, and/or intraperitoneally. They can be administered alone or in combination with anti-proliferative drugs. In one embodiment, they are administered to reduce the cancer load in the patient prior to surgery or other procedures. Alternatively, they can be administered after surgery to ensure that any remaining cancer (e.g., cancer that the surgery failed to eliminate) does not survive.

It is not intended that the present invention be limited by the particular nature of the therapeutic preparation. For example, such compositions can be provided in formulations together with physiologically tolerable liquid, gel, or solid carriers, diluents, and excipients. These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual subjects.

Such compositions are typically prepared as liquid solutions or suspensions, as injectables. Suitable diluents and excipients are, for example, water, saline, dextrose, glycerol, or the like, and combinations thereof. In addition, if desired, the compositions may contain minor amounts of auxiliary substances, such as wetting or emulsifying agents, stabilizing agents, or pH buffering agents.

Where clinical applications are contemplated, it may be necessary to prepare pharmaceutical compositions comprising proteins, antibodies, and drugs in a form appropriate for the intended application. Generally, pharmaceutical compositions may comprise an effective amount of one or more L-ASP variant or additional agents dissolved or dispersed in a pharmaceutically acceptable carrier. The phrases “pharmaceutical or pharmacologically acceptable” refers to molecular entities and compositions that do not produce an adverse, allergic, or other untoward reaction when administered to an animal, such as, for example, a human, as appropriate. The preparation of a pharmaceutical composition that contains at least one L-ASP variant isolated by the method disclosed herein, or additional active ingredient will be known to those of skill in the art in light of the present disclosure, as exemplified by Remington's Pharmaceutical Sciences, 18th Ed., 1990, incorporated herein by reference. Moreover, for animal (e.g., human) administration, it will be understood that preparations should meet sterility, pyrogenicity, general safety, and purity standards as required by the FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed., 1990, incorporated herein by reference). Except insofar as any conventional carrier is incompatible with the active ingredient, its use in the pharmaceutical compositions is contemplated.

Certain embodiments of the present invention may comprise different types of carriers depending on whether it is to be administered in solid, liquid, or aerosol form, and whether it needs to be sterile for the route of administration, such as injection. The compositions can be administered intravenously, intradermally, transdermally, intrathecally, intraarterially, intraperitoneally, intranasally, intravaginally, intrarectally, intramuscularly, subcutaneously, mucosally, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference).

The modified polypeptides may be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid. Salts formed with the free carboxyl groups can also be derived from inorganic bases, such as, for example, sodium, potassium, ammonium, calcium, or ferric hydroxides; or such organic bases as isopropylamine, trimethylamine, histidine, or procaine. Upon formulation, solutions will be administered in a manner compatible with the dosage formulation and in such amount as is therapeutically effective. The formulations are easily administered in a variety of dosage forms, such as formulated for parenteral administrations, such as injectable solutions, or aerosols for delivery to the lungs, or formulated for alimentary administrations, such as drug release capsules and the like.

Further in accordance with certain aspects of the present invention, the composition suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent, or carrier is detrimental to the recipient or to the therapeutic effectiveness of a composition contained therein, its use in administrable composition for use in practicing the methods is appropriate. Examples of carriers or diluents include fats, oils, water, saline solutions, lipids, liposomes, resins, binders, fillers, and the like, or combinations thereof. The composition may also comprise various antioxidants to retard oxidation of one or more component. Additionally, the prevention of the action of microorganisms can be brought about by preservatives, such as various antibacterial and antifungal agents, including but not limited to parabens (e.g., methylparabens, propylparabens), chlorobutanol, phenol, sorbic acid, thimerosal or combinations thereof.

In accordance with certain aspects of the present invention, the composition is combined with the carrier in any convenient and practical manner, i.e., by solution, suspension, emulsification, admixture, encapsulation, absorption, and the like. Such procedures are routine for those skilled in the art.

In a specific embodiment of the present invention, the composition is combined or mixed thoroughly with a semi-solid or solid carrier. The mixing can be carried out in any convenient manner, such as grinding. Stabilizing agents can be also added in the mixing process in order to protect the composition from loss of therapeutic activity, i.e., denaturation in the stomach. Examples of stabilizers for use in a composition include buffers, amino acids, such as glycine and lysine, carbohydrates, such as dextrose, mannose, galactose, fructose, lactose, sucrose, maltose, sorbitol, mannitol, etc.

In further embodiments, the present invention may concern the use of a pharmaceutical lipid vehicle composition that includes L-ASP variants, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Of course, compounds other than those specifically described herein that are understood by one of skill in the art as lipids are also encompassed by the compositions and methods.

One of ordinary skill in the art would be familiar with the range of techniques that can be employed for dispersing a composition in a lipid vehicle. For example, the engineered L-ASP or a fusion protein thereof may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition administered to an animal patient can be determined by physical and physiological factors, such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors, such as solubility, bioavailability, biological half-life, route of administration, product shelf life, as well as other pharmacological considerations, will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above.

### XI. COMBINATION TREATMENTS

In certain embodiments, the compositions and methods of the present embodiments involve administration of an L-ASP in combination with a second or additional therapy. Such therapy can be applied in the treatment of any disease that is associated with arginine dependency. For example, the disease may be cancer.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve administering to the cells both an L-ASP and a second therapy. A tissue, tumor, or cell can be exposed to one or more compositions or pharmacological formulation(s) comprising one or more of the agents (i.e., an L-ASP or an anti-cancer agent), or by contacting the tissue, tumor, and/or cell with two or more distinct compositions or formulations, wherein one composition provides 1) an L-ASP, 2) an anti-cancer agent, or 3) both an L-ASP and an anti-cancer agent. Also, it is contemplated that such a combination therapy can be used in conjunction with chemotherapy, radiotherapy, surgical therapy, hormone therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An L-ASP may be administered before, during, after, or in various combinations relative to an anti-cancer treatment. The administrations may be in intervals ranging from concurrently to minutes to days to weeks. In embodiments where the L-ASP is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the L-ASP and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an L-ASP is “A” and an anti-cancer therapy is “B”:

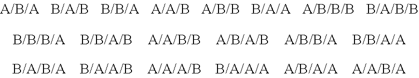

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### XII. KITS

Certain aspects of the present invention may provide kits, such as therapeutic kits. For example, a kit may comprise one or more pharmaceutical composition as described herein and optionally instructions for their use. Kits may also comprise one or more devices for accomplishing administration of such compositions. For example, a subject kit may comprise a pharmaceutical composition and catheter for accomplishing direct intravenous injection of the composition into a cancerous tumor. In other embodiments, a subject kit may comprise pre-filled ampoules of an engineered L-ASP, optionally formulated as a pharmaceutical, or lyophilized, for use with a delivery device.

Kits may comprise a container with a label. Suitable containers include, for example, bottles, vials, and test tubes. The containers may be formed from a variety of materials, such as glass or plastic. The container may hold a composition that includes an engineered L-ASP that is effective for therapeutic or non-therapeutic applications, such as described above. The label on the container may indicate that the composition is used for a specific therapy or non-therapeutic application, and may also indicate directions for either in vivo or in vitro use, such as those described above. The kit of the invention will typically comprise the container described above and one or more other containers comprising materials desirable from a commercial and user standpoint, including buffers, diluents, filters, needles, syringes, and package inserts with instructions for use.

### XIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Molecular Dynamics Simulations.

The crystal structure of E. coli L-ASN type II (PDB ID 1NNS) was used as a template for molecular simulations. In this structure, the preferred product, aspartic acid, occupies the catalytic site. Simulations included all residues (1-326) resolved in the crystal structures. Molecular transformations, assembly of the simulation cells, computational analysis of the results, and visualization were done using publicly available and custom-written scripts in Visual Molecular Dynamic (VMD) 1.8 (Humphrey et al., 1996). The substrate molecules (asparagine, glutamine) were derived from aspartic acid using the PSFGEN plugin for VMD to preserve the coordinates of the backbone and identical atoms. The N- and C-termini of INNS were modeled in the charged state. For the remaining amino acid residues, the dissociation state was estimated using ProPka (on the world wide web at propka.ki.ku.dk/) and found to be in the default state at a neutral pH level.

All water molecules resolved in the crystal structure were preserved during assembly of the starting systems. Additional water (TIP3P model) and ions (Na⁺ and Cl⁻) were added using VMD to neutralize the protein net charge of −12 and to provide a 0.13 M salt concentration, which is used in experimental studies of asparaginases. The complete system contained 19,420 protein atoms (1,304 residues), 68 (with asparagine) or 80 (with glutamine) substrate atoms, 51,302 waters, 137 sodium ions, and 125 chloride ions, for a total of approximately 174,000 atoms. The starting simulation cell was a cube with a side of 122 Å.

After minimizing the energy (1000 steps), each system was stimulated with a harmonically restrained backbone (1 kcal/mol/Å²) for 1 ns and then unrestrained for 20 ns. Simulations were performed in the NPT ensemble using the NAMD2 package (Phillips et al., 2005) with CHARMM27 force field parameters (Mackerell et al., 1998) and grid-based CMAP correction (Mackerell, 2004). The simulations used a time step of 1 fs for bonded interactions, with coordinates saved every 1 ps. The Langevin piston method (Martyna et al., 1994) was used to maintain a constant pressure of 1 atm. The temperature, set to 310.15 K, was controlled using Langevin dynamics with a coupling coefficient of 1 ps⁻¹. Periodic boundary conditions were used to eliminate surface effects. The particle mesh Ewald method (Darden et al., 1993), with a real-space cutoff distance of 10 Å and grid width of 1 Å, was used to compute electrostatic energies. The switching distance for non-bonded electrostatics and van der Waals interactions was 8.5 Å, with force update time steps of 4 and 2 fs, respectively. The High Performance Computer Cluster at the University of Maryland, College Park provided computational time.

Analysis of Simulation Results.

Contact counting was performed every 10 ps throughout the simulation. The count, averaged over the last 10 ns (from 10 to 20 ns), was used to estimate the probability of enzyme residue location within the first coordination shell of the substrate. Those contacts reflect the orientation and chemical interaction of the substrate in the enzyme's binding pocket. The width of the first contact shell (3 Å) was chosen to approximate the average position of the first minimum in the radial distribution function between contacting heavy atoms of ligand (substrate and water) and enzyme. The number of enzyme-water and enzyme-substrate contacts reached a constant average value after the first 8 ns of simulation time, suggesting that the structures used were representative of the thermal state of the system.

Compounds and Plasmids.

ELSPAR® (Escherichia coli L-Asp) was purchased from Lundbeck Pharmaceuticals. The gene coding for E. coli L-Asparaginase II (ansB; referred to herein as L-ASP) was polymerase chain reaction-amplified from genomic DNA of E. coli TOP 10 strain (Invitrogen). To facilitate purification of recombinant proteins, a 6× histidine tag was incorporated in the forward primers (SEQ ID NOs: 1 and 2).

PCR products were then cloned into the NcoI restriction site of the pET-22b(+) expression vector (Novagen) using the IN-FUSION® HD ECODRY™ Cloning Kit (Clontech). The resulting plasmid contained an N-terminal pelB leader peptide, a 6× histidine tag, and the gene sequence coding for mature E. coli L-ASP (excluding the signal sequence encoded by the first 22 amino acids of the full-length sequence). His-tagged W. succinogenes L-ASP expression vector was also generated by cloning a PCR product amplified from its genomic DNA (ATCC) into the NcoI site of pET-22b(+) vector (primers 162 and 163; SEQ ID NOs: 3 and 4). To generate expression vectors of E. coli L-ASP Q59 mutants, two PCR reactions were performed using the wild-type (WT) expression vector as the template. One reaction amplified the L-ASP fragment including mutated Q59 (primers 112 and 113; SEQ ID NOs: 5 and 6), and the other reaction used deletion PCR to amplify the expression vector sequence (primers 114 and 115; SEQ ID NOs: 7 and 8). Those two PCR products were then ligated to generate the expression vectors of Q59 mutants using IN-FUSION® cloning. The primers for the first PCR reaction included a degenerate NNS codon (N can be A, T, C, or G; S can be C or G) at the site corresponding to Q59 of full-length L-ASP. All mutations were verified by DNA Sanger sequencing.

Determination of Asparaginase and Glutaminase Enzyme Activity.

Asparaginase enzyme activity was measured using an established colorimetric asparaginase assay. The assay, which uses L-aspartic acid β-hydroxamate (AHA) as a substrate (Wehner et al., 1992), was modified as follows. 25 μL of diluted bacterial culture supernatant, or purified enzyme, in activity buffer (50 mM Tris-HCl, pH 8.0) was mixed with 25 μL of 10 mM AHA in a 96-well PCR plate in triplicate. After incubation at 37° C. for 8 min, 50 μL color reagent (2% 8-hydroxyquinoline in ethanol with 1 M Na₂CO₃=1:3 (v/v)) was added to each well, and the plate was heated at 85° C. for 90 s. The plate was then cooled at 4° C. for 5 min, and the reaction mixture was transferred to 1 well of a 96-well flat bottom pyrostyrene plate (Corning) for measurement of absorbance at 705 nM. The asparaginase activity of purified, recombinant L-ASP was calculated in International Units (IU) based on the ELSAPR® standard curve.

Glutaminase enzyme activity was measured using a Glutamate Assay kit (AbCam) according to the manufacturer's instructions. One IU of glutaminase activity is defined as the amount of enzyme required to generate 1 μmol of glutamate per minute at pH 8.0 and 37° C. For measurement of the glutaminase activity of recombinant L-ASP, 200 μL of reaction mixture containing enzyme equivalent to 0.2 IU of asparaginase activity, 50 mM Tris-HCl, pH 8.0, and 200 μM glutamine was incubated at 37° C. After 60 min, 50 μL of reaction was used for measurement of the amount of the product, glutamate.

Asparaginase and glutaminase activities were also determined using a highly sensitive liquid chromatography-mass spectrometry (LC-MS)/MS assay (Purwaha et al., 2014). Limits of detection for asparagine, aspartic acid, glutamine, and glutamic acid were 250, 150, 16, and 22 nM, respectively.

Mutagenesis, Expression, Purification, and Screening of L-ASP Recombinant Enzymes.

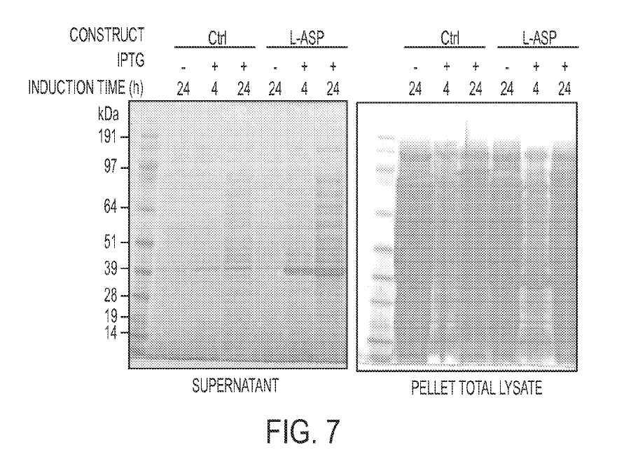

L-ASP expression vectors with a pelB leader sequence (Khushoo et al., 2004) were transformed into the E. coli BLR (DE3) strain (Novagen) (FIG. 7). Transformed cells were grown in LB broth (Sigma) supplemented with ampicillin (100 μg/ml) at 37° C., with shaking at 220 rpm. Inoculation cultures at 10% (v) were grown for 3 h then induced with 0.1 mM isopropyl β-D-1-thiogalactopyranoside (IPTG) for 16 h to express extracellular L-ASP. The supernatant was collected by centrifuging cultures at 5,000 g for 15 min at 4° C. and passing through a 0.22-μm EXPRESS® PLUS Filter Unit (Millipore). Ni-NTA resin (Qiagen) was used for purification of recombinant L-ASP according the manufacturer's instructions. Fractions containing L-ASP were identified by SDS-PAGE analysis, pooled, and dialyzed against 50 mM Tris-HCl, pH 8.0, using Dialysis Cassette G2 with a 10 kDa MW cut-off (Thermo). The purified protein solution was concentrated using a centrifugal filter unit with 10 kDa MW cut-off (Millipore). Protein concentration was determined using the BCA Protein Assay (Thermo).

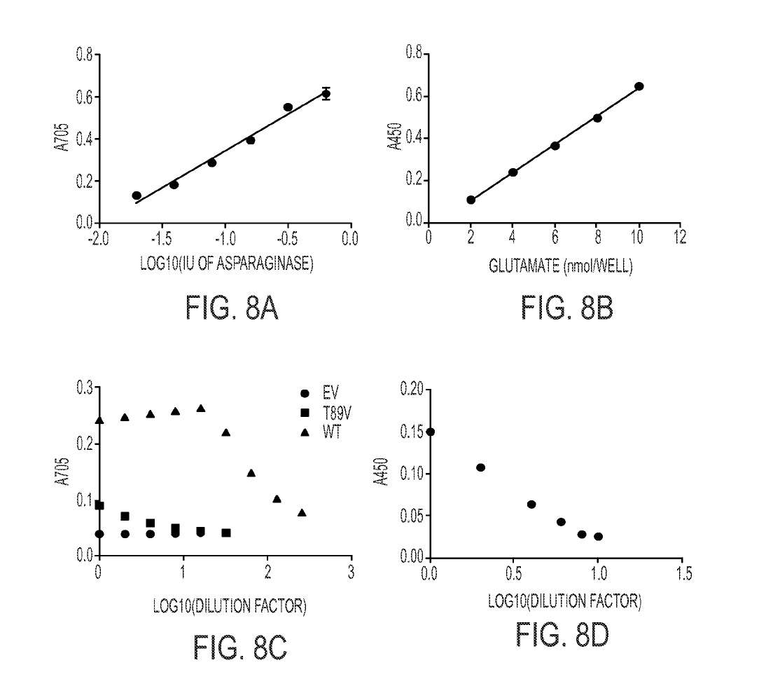

Asparaginase and glutaminase activities of L-ASP were screened using the aforementioned colorimetric assays. The enzymatically inactive T89V mutant (Palm et al., 1996) and empty expression vector served as negative controls. Both asparaginase and glutaminase enzymatic activities were correlated with enzyme concentration (FIG. 8). Hence, a rapid screening procedure was developed for measuring the asparaginase and glutaminase activities of L-ASP without protein purification. To validate the method for use on unpurified supernatants, parallel assays were performed after purification of the L-ASP mutant proteins.

RNA Interference (RNAi) and Cell Proliferation Assays.

All mammalian cell lines were maintained in RPMI 1640 (HyClone) with 5% fetal bovine serum (HyClone) and 2 mM L-glutamine (HyClone), as described previously (Lorenzi et al., 2006). Small interfering RNA (siRNA) assays were performed as described previously (Lorenzi et al., 2006) with the following modifications: 96-well culture plates used final 5 nM siRNA, 0.10 μL of INTERFERIN™ (PolyPlus Transfection), and 1500 cells per well in a 100 μL total volume. Cell proliferation was assessed using CELLTITER-BLUE® or 3-(4,5-dimethylthiazol-2-yl)-5-(3-carboxymethoxyphenyl)-2-(4-sulfophenyl)-2H-tetrazolium, inner salt (MTS) assay (Promega) according to the manufacturer's instructions, and L-ASP 50% effective concentration (EC₅₀) values were determined using GraphPad Prism 6 (GraphPad Software) as described previously (Lorenzi et al., 2006). All mammalian cell lines were tested for Mycoplasma using the MycoAlert assay (Lonza) at the commencement of this study and found to be negative. In addition, DNA fingerprints were obtained for all cell lines and were concordant with those previously reported (Lorenzi et al., 2009).

Detection of ASNS Protein.

For measurement of ASNS protein, total cell protein was extracted using bicine/CHAPs lysis buffer (Protein Simple). Twenty micrograms of total protein per lane was electrophoresed in sodium dodecyl sulfate (SDS)-polyacrylamide, transferred to Immun-Blot polyvinylidene difluoride membrane (Bio-Rad), and probed with antibodies against ASNS or β-actin (Sigma) as previously reported (Lorenzi et al., 2006). Relative ASNS and β-actin expression levels were quantified using ImageJ.

Kinetic Competition Analysis of L-ASP Mutants.

The kinetics of asparagine and glutamine catabolism by L-ASP were analyzed using a reaction containing 100 μM asparagine, 1600 μM glutamine, 23 mM Tris-HCl at pH 8.5 and either WT L-ASP (15 nM) or its Q59L mutant (200 nM). Aliquots (30 μL) were withdrawn from 500 μL reaction mixtures over a 60-min time course. The reaction was immediately quenched by adding the aliquot to 120 μL of dry ice-cooled methanol containing 10 μM ¹³C ¹⁵N-labeled internal standards of asparagine, aspartate, glutamine, and glutamate. Those quenched aliquots were then analyzed by LC-MS/MS.

### Example 2

### Molecular Dynamics of L-ASP

To guide mutagenesis experiments aimed at creating a glutaminase-deficient mutant, 20-ns MD simulations of WT L-ASP bound with asparagine or glutamine were performed. Preferential orientations and contacts of the two substrates within the lining of the L-ASP catalytic cleft were compared and critical differences in how each substrate is coordinated were identified. Analysis of enzyme-substrate contact times served as a basis for identifying the most promising mutagenesis target.

Both substrates changed positions within ˜200 to 300 ps compared with the crystallographic orientation of aspartate. That re-orientation occurred in all four enzyme-binding pockets of the tetramer, clearly establishing a different preference for asparagine and glutamine compared with the product, aspartate. The re-orientation could be attributed to the fact that both substrates include uncharged amide side chains rather than the negatively charged carboxylate moiety of the product. Additional differences from crystal structures, which are formed by tightly packed enzymes under low hydration, are expected due to simulation conditions at physiological protein concentrations, ion concentrations, and temperature. The dynamics of equilibration in the course of 20-ns simulations is shown in FIG. 15. As illustrated in FIG. 15B, the probability of contact between glutamine side-chain amide oxygen (—CONH₂, labeled “OE1”) and the enzyme diminished in the interval between 6 and 8 ns, whereas probabilities of other interactions fluctuated around stable mean values.

The enzyme residues and specific atoms forming the first shell around asparagine and glutamine are listed in Table 1. The criterion for inclusion in the first shell was proximity of <3 Å for a duration of >1% of the entire simulation time. Probabilities were averaged over all four of the enzyme's binding sites. Table 1 suggests that both substrates are coordinated by essentially the same sets of atoms. Importantly, side-chain amide oxygens of asparagine and glutamine approached the catalytic hydroxyl (—OH) group of threonine T12 with probabilities of only 3% and 1%, respectively (Table 1). In contrast, their α-carboxylate groups (—COO⁻) contacted the catalytic —OH of T12 for much larger fractions of time (77% and 48%, respectively).

The differences between substrate contacts (Table 1; right-most columns) indicated a decreased probability of contact between glutamine and almost every residue in the enzyme catalytic site. Notable exceptions were the backbone α-amino groups (—NH) of glutamine Q59 and threonine T89, and the side-chain amine (—NH₃⁺) of lysine K162. The most notable difference between asparagine and glutamine substrates, in fact, was the mode of interaction with Q59 (FIGS. 1A-B). Increased interaction of the glutamine substrate's α-carboxyl with the Q59 backbone amide coincided with reduced interaction with the catalytic T12 residue. As a consequence, the α-carboxyl of glutamine re-oriented toward the backbone amide of T89, and the side-chain amide of glutamine lost contact with both the backbone amide and side-chain hydrogen bond donors of T89. As T89 typically coordinates the amide group of asparagine, the lost contact with glutamine appeared to be of significant importance.

Those results suggested that residue Q59 of L-ASP would be a more promising site for mutagenesis than K162 or T89. The latter residue plays an important catalytic role in the second stage of the reaction (hydrolysis of the aspartyl-enzyme bond) and, therefore, should not be mutated (Palm et al., 1996). Similarly, K162 is involved in electrostatic stabilization of several charges in the catalytic cleft (Palm et al., 1996; Verma et al., 2007) and, thus, should not be mutated. Q59, on the other hand, coordinates the backbone groups but not the side chains of both substrates. Therefore, mutations in Q59 would be less likely to render the enzyme completely inactive.

TABLE 1
Probability of specific contacts between polar groups of the enzyme and substrates.

| | | Asn | | | | Gln | | | | Gln-Asn | | | |
|---|---|---|---|---|---|---|---|---|---|---|---|---|---|
| | | Backbone | | | | Backbone | | | | Backbone | | | |
| | | | OT1 | Side Chain | | | OT1 | Side Chain | | | OT1 | Side Chain | |
| Residue | Atom | N | OT2 | OD1 | ND2 | N | OT2 | OD1 | ND2 | N | OT2 | OD1 | ND2 |
| T12 | N | | 0.80 | | | | 0.17 | 0.06 | | | −0.63 | 0.06 | |
| | OG1 | | 0.77 | 0.03 | 0.08 | | 0.48 | 0.01 | 0.05 | | −0.29 | −0.02 | −0.03 |
| Y25 | OH | | | | | 0.00 | | | 0.01 | 0.00 | | | 0.01 |
| G57 | CA | | | | | | 0.02 | | | | 0.02 | | |
| S58 | N | | 0.68 | | | | 0.65 | | | | −0.03 | | |
| | OG | 0.00 | 0.95 | | | 0.00 | 0.86 | | | 0.00 | −0.09 | | |
| Q59 | N | | | | | | 0.27 | | | | 0.27 | | |
| | OE1 | 0.61 | 0.03 | | | 0.59 | 0.01 | | | −0.02 | −0.01 | | |
| G88 | CA | | 0.01 | 0.01 | | | 0.01 | 0.00 | | | 0.00 | 0.00 | |
| T89 | N | | 0.06 | 0.74 | | | 0.31 | 0.14 | | | 0.25 | −0.60 | |
| | OG1 | | 0.05 | 0.70 | 0.01 | | 0.07 | 0.01 | 0.04 | | 0.03 | −0.69 | 0.03 |
| D90 | N | | 0.01 | | | | 0.04 | | | | 0.03 | | |
| | OD1 | | 0.00 | | | 0.05 | | 0.00 | 0.00 | 0.05 | 0.00 | 0.00 | 0.00 |
| | OD2 | 0.99 | 0.00 | | | 0.98 | 0.01 | | 0.00 | −0.01 | 0.01 | | 0.00 |
| A114 | O | | | 0.11 | 0.55 | | | 0.00 | 0.13 | | | −0.11 | −0.43 |
| M115 | O | | | | 0.00 | | | | 0.04 | | | | 0.04 |
| K162 | NZ | | | | | | | 0.12 | 0.00 | | | 0.12 | 0.00 |
| N246 | ND2 | | | | | | | 0.02 | 0.00 | | | 0.02 | 0.00 |
| E283 | OE1 | 0.85 | | 0.01 | 0.00 | 0.30 | | | | −0.55 | | −0.01 | 0.00 |
| | OE2 | 0.55 | | 0.00 | 0.00 | 0.28 | | | | −0.27 | | 0.00 | 0.00 |

Values indicate the fractions of time that residues from the row and the column spend within 3 Å of each other. Residues with a total contact probability above 1% are shown in bold. Enzyme atom labeling scheme: backbone amine (N), α-carbon (CA), side-chain oxygens (OG = γ -oxygen; OE = ε-oxygen; OD = δ-oxygen), side chain hydroxyl (OH), backbone α-carbonyl (O), side-chain amine (NZ, ND2). Positive values indicate higher frequency of enzyme-substrate contacts when Asn is replaced by Gln; negative values indicate the opposite.

### Example 3

### Characterization of L-ASP Q59 Mutants

Site-directed mutagenesis was performed to obtain Q59 variants of the enzyme. L-ASP expression vectors, coding for all 20 possible amino acids at position 59, were transformed into E. coli BL-21. All mutants except Q59C and Q59S were expressed and secreted into the culture medium as efficiently as the WT protein (FIG. 2A). Subsequent kinetic screening using colorimetric assays indicated that the mutants exhibited a spectrum of asparaginase activity ranging from 0% to 80% of WT, with a median of 12% (FIG. 2B). The Q59 mutants also exhibited a spectrum of glutaminase activity ranging from 0% to 60% of WT, but the median was just 2% of WT glutaminase activity (FIG. 2C), suggesting that Q59 is indeed more important for glutaminase activity than for asparaginase activity.

To exclude the possibility that endogenous E. coli L-ASP was contributing to the asparaginase and glutaminase activities measured on non-purified supernatants, the Q59L, Q59F, Q59D, Q59E, Q59H, and Q59N mutants were purified and it was found that they yielded results consistent with the initial screen (FIGS. 2D-E). Thus, the screening of non-purified supernatants was quantitatively reliable (FIG. 8). FIG. 2F illustrates glutaminase:asparaginase ratios of the purified mutants. Q59L and Q59F exhibited the smallest glutaminase:asparaginase ratios, with almost undetectable glutaminase activity and 80% and 25% of WT asparaginase activity, respectively. Next, a sensitive LC-MS/MS assay was used to confirm that Q59L exhibits negligible glutaminase activity as indicated by measurement of glutamic acid after incubation with glutamine for 1 h (FIG. 9). For comparison, Q59L exhibited even lower glutaminase activity than that of W. succinogenes L-ASP (FIG. 9C), which was previously reported, using less sensitive methods, to exhibit very low glutaminase activity (Distasio et al., 1976; Distasio et al., 1977; Lubkowski et al., 1996). Q59H exhibited the largest ratio of glutaminase:asparaginase activity.

### Example 4

### Kinetic Characterization of Q59L L-ASP

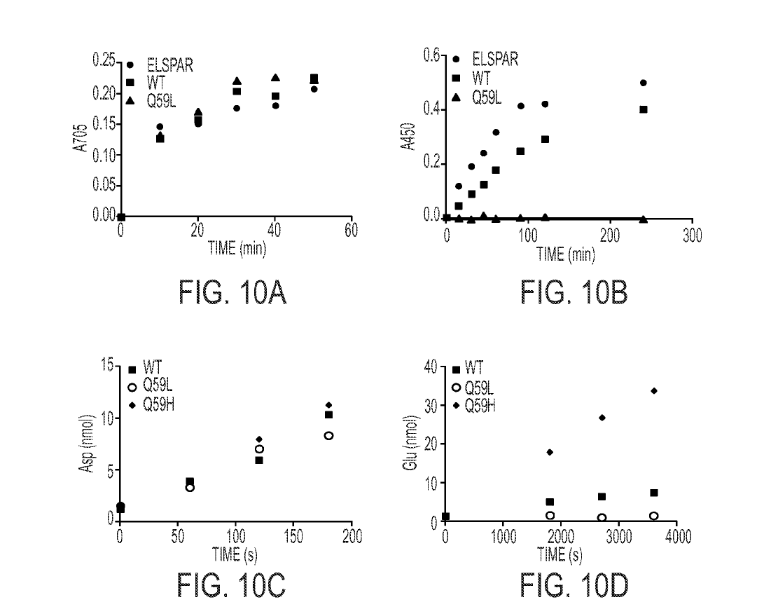

ELSPAR®, the clinical variant of L-ASP, was compared with WT L-ASP and Q59 L-ASP with respect to their kinetics of asparagine and glutamine deamidation. Using optimized steady-state reaction conditions, the initial rate of product formation (v₀) measured by colorimetric asparaginase assay was found to be equivalent for all three enzymes when used at equivalent asparaginase concentrations (FIGS. 10A-B). The corresponding glutaminase activity of WT L-ASP was slightly less than that of ELSPAR®, and the glutaminase activity of Q59L was not detected.

However, the colorimetric assays may be misleading for kinetic analysis because they are based on derivatives of the amino acid substrates and products, rather than the amino acids themselves. Hence, a LC-MS/MS assay was used for more reliable and sensitive analysis. First, to compare the glutaminase activities of the L-ASP variants, it was determined that 10, 20, and 60 nM concentrations of WT, Q59L, and Q59H L-ASP, respectively, exhibited nearly identical asparaginase initial reaction rates (˜4.8×10⁻² nmol/s) (FIG. 10C). Using the same ratio (40, 80, and 240 nM) did not yield equivalent glutaminase activities; initial reaction rates were 1.7×10⁻³, <9.8×10⁻⁵ (near the assay detection limit), and 9.0×10⁻³ nmol/s, respectively (FIG. 10D), indicating that Q59L exhibits undetectable glutaminase activity.

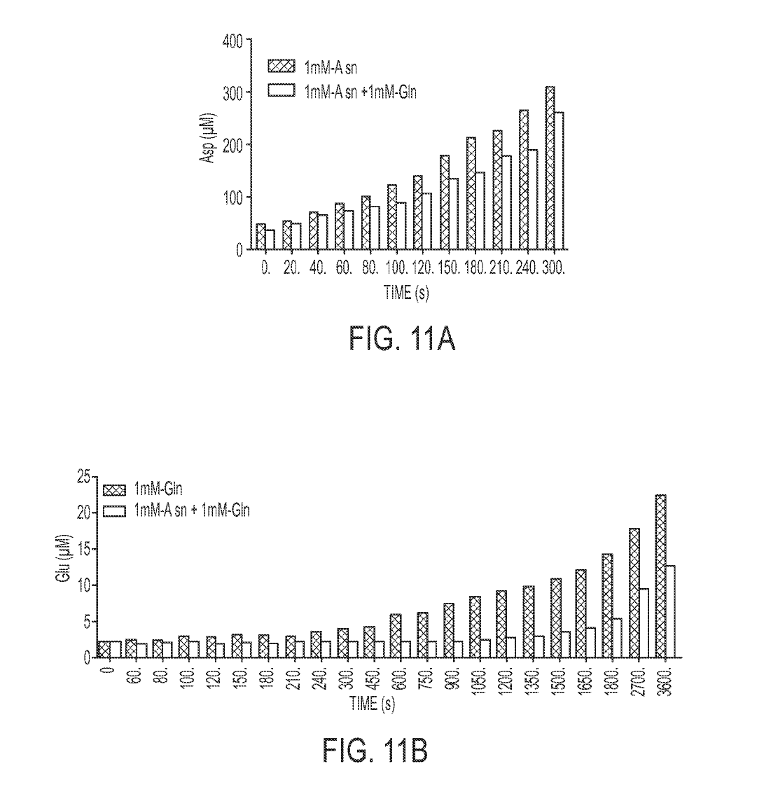

The substrate competition kinetics were analyzed using physiologically relevant concentrations of asparagine and glutamine. FIG. 3 shows the resulting time course of asparagine depletion and glutamate formation in single-substrate reactions (solid symbols) or in a mixture of the two substrates (open symbols). WT L-ASP completely degraded pure 100 μM asparagine in a linear fashion within ˜500 s (FIG. 3A, solid circles), whereas the presence of 1600 μM glutamine delayed asparagine degradation to ˜600 s (FIG. 3A, open circles). In the single reaction with glutamine, glutamate was formed immediately and linearly over 1200 s (FIG. 3B, solid triangles), but it was not detected in the mixture until ˜600 s (FIG. 3B, open circles). As asparagine was almost fully depleted at 600 s in the reaction with both substrates (FIG. 3A, open circles) and glutamate did not begin to appear until that point, the data suggest a strong kinetic preference of WT L-ASP for asparagine. Additional competition experiments in which both substrates were used at 1 mM also yielded a time lag in the appearance of reaction products aspartic acid and glutamic acid (FIGS. 11A-B). Q59L, to the contrary, did not exhibit measurable glutaminase activity (FIG. 3B). Moreover, 1600 μM glutamine did not inhibit the asparaginase activity of Q59L, indicating that glutamine is not strongly bound (or deamidated) by Q59L.

### Example 5

### Anticancer Activity of L-ASP Q59 Mutants

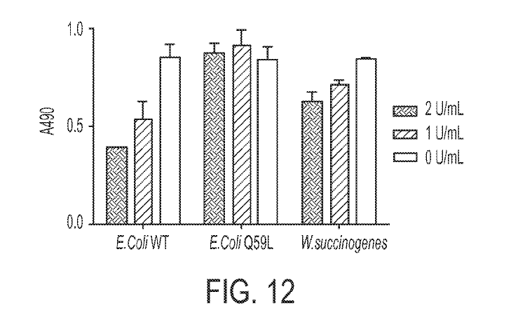

To investigate the contribution of glutaminase activity to the anticancer activity of L-ASP, purified WT, Q59L, or Q59F L-ASP was used to treat six leukemia lines (CCRF-CEM, SR, MOLT-4, K562, NALM-6, and REH) (FIGS. 4A-F) and two ovarian cancer lines (OVCAR-8 and SK-OV-3) (FIGS. 4G-H). Dosages were scaled to match asparaginase specific activity (IU/mg enzyme). The purified WT enzyme yielded anticancer activity comparable to that of ELSPAR® (FIGS. 4A-B). In contrast, glutaminase-deficient Q59L and Q59F did not exhibit measurable anticancer activity against any of the eight lines, even at the highest dose, 32 U/mL, indicating that glutaminase activity is essential to the anticancer activity of L-ASP in those cell lines. In further support of that conclusion, W succinogenes L-ASP, which exhibits weak glutaminase activity (FIG. 9C), retained only weak anticancer activity (FIG. 12). In contrast to the glutaminase-deficient Q59L and Q59F mutants, Q59 mutants that retained glutaminase activity (Q59D, Q59E, Q59H, Q59N) exhibited anticancer activity comparable to that of WT L-ASP (FIGS. 4I-J). Overall, the results indicate that the glutaminase activity of L-ASP contributes to anticancer activity in the cell lines listed in this section.

### Example 6

### ASNS Mediates Resistance to L-ASP Q59 Mutants

A negative correlation between the anticancer activity of L-ASP and the expression of ASNS has been reported (Lorenzi et al., 2008; Lorenzi et al., 2006; Aslanian et al., 2001). To determine whether ASNS also mediates resistance to glutaminase-deficient Q59 mutants, ASNS western blot analysis was performed before and after treatment of cell lines that were found to be insensitive to the glutaminase-deficient Q59L and Q59F L-ASP mutants. As expected, ASNS was extensively upregulated in all of the cell lines tested (FIG. 4K). Next, western blot analysis of ASNS was performed following treatment of OVCAR-8 cells with selected Q59 mutants. ASNS was also upregulated by all six mutants tested, albeit to a different extent for each mutant (FIG. 4L). Interestingly, high-glutaminase Q59H induced the lowest extent of ASNS upregulation, suggesting that the extent of ASNS upregulation may be suppressed by the glutaminase activity of L-ASP.

A functional genomics approach, RNAi, was used to test whether ASNS upregulation mediates resistance to glutaminase-deficient L-ASP. ASNS siRNA treatment resulted in highly effective knockdown of ASNS protein in OVCAR-8 cells (FIG. 5A). ASNS siRNA potently sensitized OVCAR-8 cells to glutaminase-deficient Q59L (FIG. 5B). Moreover, Q59L and Q59F exerted potent anticancer activity against the leukemia cell lines Sup-B15 and RS4;11 (FIGS. 5C-D), which did not express detectable levels of ASNS protein before or after L-ASP treatment (FIG. 5E). Accordingly, the Sup-B15 and RS4;11 cells lines are referred to as “ASNS-negative.” Notably, the anticancer activity of Q59L against the Sup-B15 line (EC₅₀=1.4×10⁻⁴ U/mL) and RS4;11 (EC₅₀=1.3×10⁻³ U/mL) was greater than or equal to the anticancer activity of Q59L against the ASNS siRNA-treated OVCAR-8 line (EC₅₀=1.1×10⁻³ U/mL). This represents a degree of anticancer activity that reflects the greatest in vitro L-ASP potency observed to date while performing measurements on over 70 cell types (Lorenzi et al., 2008; Scherf et al., 2000; Lorenzi et al., 2006). In summary, these results demonstrate that ASNS-negative cancer cell lines are hypersensitive to asparaginase activity alone (i.e., asparagine depletion without glutamine depletion).

### Example 7

### Anticancer Activity of WT, Q59L, and Q59H L-ASP

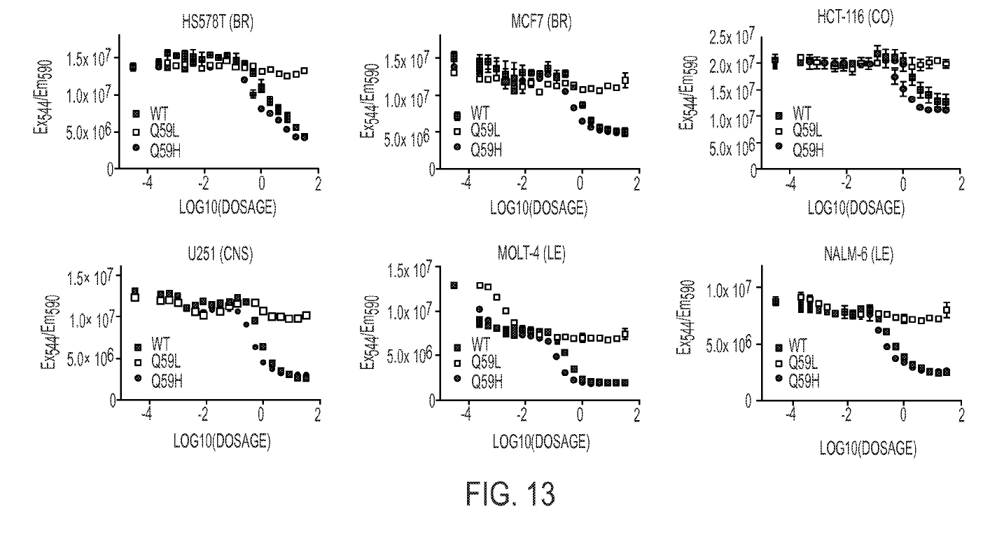

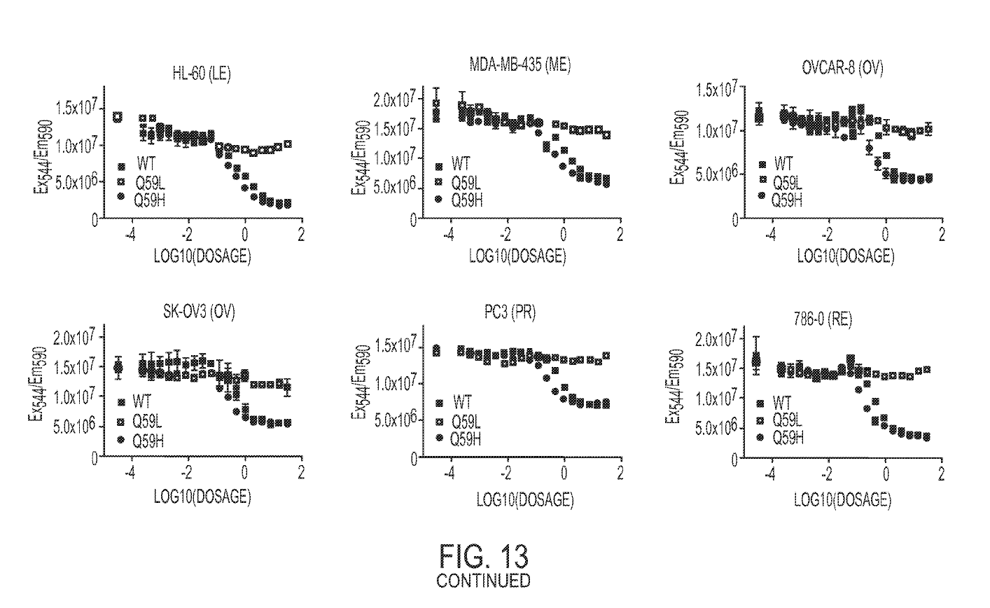

Twelve cell lines were treated with a range of WT (closed squares), Q59L (open squares), or Q59H L-ASP (closed circles) concentrations for 48 h then assayed with CELLTITER-BLUE® (FIG. 13). These data show that the Q59H L-ASP mutant has anticancer activity against a variety of cancer cell types including breast cancer (BR), colorectal cancer (CO), central nervous system cancer (CNS), leukemia (LE), melanoma (ME), ovarian cancer (OV), prostate cancer (PR), and renal cancer (RE).

### Example 8

### Anticancer Activity of Q59L L-Asparaginase in an Acute Lymphoblastic Leukemia Mouse Model

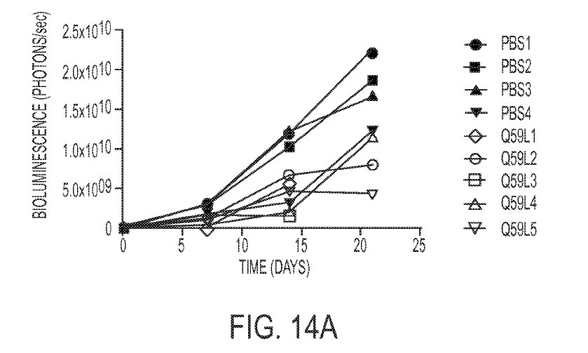

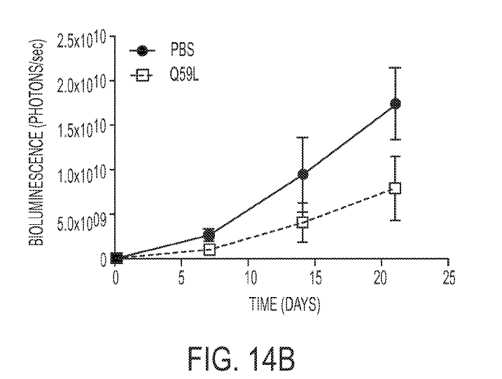

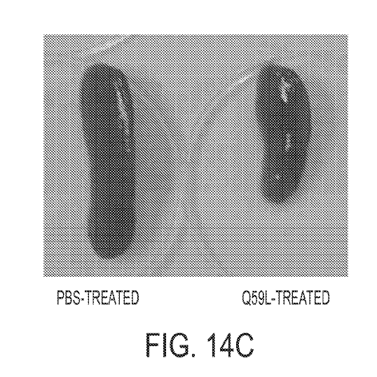

NOD/SCID/IL2Rgamma knockout (NSG) mice were injected with Sup-B15/luciferase cells and treated with PBS or Q59L starting two weeks after injection (i.p., 3 times a week for 3 weeks). Luciferase activity (i.e., tumor burden) of Q59L-treated and control mice was measured over the course of the experiment (FIGS. 14A-B). Q59L treatment decreased tumor proliferation (FIGS. 14A-B) and prevented leukemia infiltration of the spleen (FIG. 14C).

### Example 9

### Characterization of L-ASP S58 Mutants

Site-directed mutagenesis was performed to obtain S58 variants of the enzyme. L-ASP expression vectors, coding for all 20 possible amino acids at position 58, were transformed into E. coli BL-21. Subsequent kinetic screening using colorimetric assays indicated that the mutants exhibited a spectrum of asparaginase activity ranging from 0% to 110% of WT, with a mean of 21.12% (FIG. 15A). The S58 mutants also exhibited a spectrum of glutaminase activity ranging from 0% to 20% of WT, but the mean was just 7.97% of WT glutaminase activity (FIG. 15B), suggesting that S58 is more important for glutaminase activity than for asparaginase activity. S58G, S58T, and S58A exhibited the smallest glutaminase:asparaginase ratios, with undetectable glutaminase activity and 99.21%, 103.84%, and 99.21% of WT asparaginase activity, respectively. These mutants therefore represent promising candidates for improving the therapeutic index of L-ASP.

### Example 10

### Anticancer Activity of L-ASP S58 Mutants

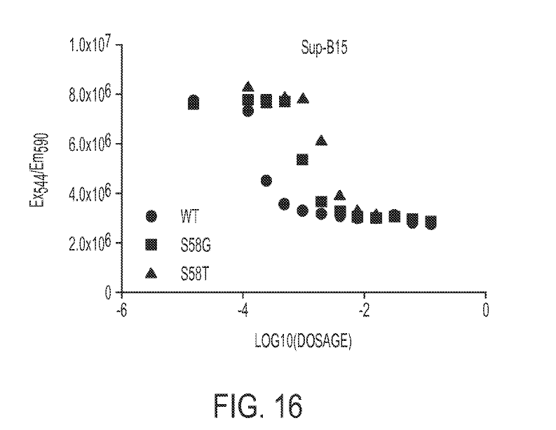

S58G and S58T exerted potent anticancer activity against the leukemia cell line Sup-B15 (FIG. 16), which did not express detectable levels of ASNS protein before or after L-ASP treatment (FIG. 5E). Accordingly, the Sup-B15 cells line is referred to as “ASNS-negative.” Notably, the anticancer activity of S58G and S58T against the Sup-B15 line were EC₅₀=1.0×10⁻³ U/mL and 2.6×10⁻³ U/mL, respectively. Thus, glutaminase-free S58 mutants of L-ASP exhibit potent anticancer activity against ASNS-negative cell types.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,192,869
U.S. Pat. No. 4,321,259
U.S. Pat. No. 4,473,563
U.S. Pat. No. 4,478,824
U.S. Pat. No. 4,652,449
U.S. Pat. No. 4,752,586
U.S. Pat. No. 4,870,287
U.S. Pat. No. 4,874,690
U.S. Pat. No. 4,931,276
U.S. Pat. No. 5,043,261
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,846,945
U.S. Pat. No. 5,889,155
U.S. Pat. Publn. 2009/0304666
Aslanian et al., Asparagine synthetase expression alone is sufficient to induce 1-asparaginase resistance in MOLT-4 human leukaemia cells. Biochem. J., 357(Pt 1):321-328, 2001.
Asrani et al., Glutamine supplementation in acute pancreatitis: a meta-analysis of randomized controlled trials. Pancreatology, 13(5):468-474, 2013.
Austin-Ward and Villaseca, Revista Medica de Chile, 126(7):838-845, 1998.
Ausubel et al., Current Protocols in Molecular Biology, Greene Publishing Associates and Wiley Interscience, N.Y., 1994.
Bukowski et al., Clinical Cancer Res., 4(10):2337-2347, 1998.
Bussey et al., Integrating data on DNA copy number with gene expression levels and drug sensitivities in the NCI-60 cell line panel. Mol. Cancer Ther., 5(4):853-867, 2006.
Cantor et al., Therapeutic enzyme deimmunization by combinatorial T-cell epitope removal using neutral drift. Proc. Natl. Acad. Sci. USA, 108:1272-1277, 2011.
Christodoulides et al., Microbiology, 144(Pt 11):3027-3037, 1998.
Darden et al., Particle Mesh Ewald—an N·Log(N) method for Ewald sums in large systems. J. Chem. Phys., 98:10089-10092, 1993.
Davidson et al., J. Immunother., 21(5):389-398, 1998.
Derst et al., Engineering the substrate specificity of Escherichia coli asparaginase II. Selective reduction of glutaminase activity by amino acid replacements at position 248. Protein Sci., 9(10):2009-2017, 2000.
Distasio et al., Purification and characterization of L-asparaginase with anti-lymphoma activity from Vibrio succinogenes. J. Biol. Chem., 251(22):6929-6933, 1976.
Distasio et al., Antilymphoma activity of a glutaminase-free L-asparaginase of microbial origin. Proc. Soc. Exp. Biol. Med., 155(4):528-531, 1977.
Distasio et al., Glutaminase-free asparaginase from vibrio succinogenes: an antilymphoma enzyme lacking hepatotoxicity. Int. J. Cancer, 30(3):343-347, 1982.
Dufour et al., Pancreatic tumor sensitivity to plasma L-asparagine starvation. Pancreas, 41(6):940-948, 2012.
Durden and Distasio, Characterization of the effects of asparaginase from Escherichia coli and a glutaminase-free asparaginase from Vibrio succinogenes on specific ell-mediated cytotoxicity. Int. J. Cancer, 27(1):59-65, 1981.
Durden et al., Kinetic analysis of hepatotoxicity associated with antineoplastic asparaginases. Cancer Res., 43(4):1602-1605, 1983.
Ehsanipour et al., Adipocytes cause leukemia cell resistance to L-asparaginase via release of glutamine. Cancer Res., 73(10):2998-3006, 2013.
Fine et al., A genome-wide view of the in vitro response to 1-asparaginase in acute lymphoblastic leukemia. Cancer Res., 65(1):291-299, 2005.
Fumarola et al., Glutamine deprivation-mediated cell shrinkage induces ligand-independent CD95 receptor signaling and apoptosis. Cell Death Differ., 8(10):1004-1013, 2001.
Gesto et al., Unraveling the enigmatic mechanism of L-asparaginase II with QM/QM calculations. J. Am. Chem. Soc., 135(19):7146-7158, 2013.
Hanibuchi et al., Int. J. Cancer, 78(4):480-485, 1998.
Harkki et al., BioTechnology, 7:596-603, 1989.
Haskell and Canellos, 1-asparaginase resistance in human leukemia-asparagine synthetase. Biochem. Pharmacol., 18(10):2578-2580, 1969.
Hellstrand et al., Acta Oncologica, 37(4):347-353, 1998.
Hollander, Front. Immun., 3:3, 2012.
Hopwood et al., In: Genetic Manipulation of Streptomyces, A Laboratory Manual, The John Innes Foundation, Norwich, Conn., 1985.
Horowitz et al., Asparagine synthetase activity of mouse leukemias. Science, 160(3827):533-535, 1968.
Hui and Hashimoto, Infection Immun., 66(11):5329-5336, 1998.
Humphrey et al., VMD: visual molecular dynamics. J. Mol. Graph., 14(1):33-38, 1996.
Hutson et al., Amino acid control of asparagine synthetase: relation to asparaginase resistance in human leukemia cells. Am. J. Physiol., 272(5 Pt 1):C1691-C1699, 1997.
Jenkins and Perlin, Severe hepatotoxicity from Escherichia coli L-asparaginase. J. Natl. Med. Assoc., 79(7):775, 779, 1987.
Kafkewitz and Bendich, Enzyme-induced asparagine and glutamine depletion and immune system function. Am. J. Clin. Nutr., 37(6):1025-1030, 1983.
Khushoo et al., Extracellular expression and single step purification of recombinant Escherichia coli L-asparaginase II. Protein Expr. Purif., 38(1):29-36, 2004.
Kitoh et al., The inhibition of lymphocyte blastogenesis by asparaginase: critical role of glutamine in both T and B lymphocyte transformation. Acta Paediatr. Jpn., 34(6):579-583, 1992.
Labrou et al., Structure-function relationships and clinical applications of L-asparaginases. Curr. Med. Chem., 17(20):2183-2195, 2010.
Leslie et al., Expression levels of asparagine synthetase in blasts from children and adults with acute lymphoblastic leukaemia. Br. J. Haematol., 132(6):740-742, 2006.
Lordanescu, J. Bacteriol, 12:597 601, 1975.
Lorenzi et al., N-methylpurine DNA glycosylase and 8-oxoguanine dna glycosylase metabolize the antiviral nucleoside 2-bromo-5,6-dichloro-1-(beta-D-ribofuranosyl)benzimidazole. Drug Metab. Dispos., 34(6):1070-1077, 2006.
Lorenzi et al., Asparagine synthetase as a causal, predictive biomarker for L-asparaginase activity in ovarian cancer cells. Mol. Cancer Ther., 5(11):2613-2623, 2006.
Lorenzi et al., Asparagine synthetase is a predictive biomarker of L-asparaginase activity in ovarian cancer cell lines. Mol. Cancer Ther., 7(10):3123-3128, 2008.
Lorenzi et al., DNA fingerprinting of the NCI-60 cell line panel. Mol. Cancer Ther., 8(4):713-724, 2009.
Lubkowski et al., Crystal structure and amino acid sequence of Wolinella succinogenes L-asparaginase. Eur. J. Biochem., 241(1):201-207, 1996.
Mackerell et al., All-atom empirical potential for molecular modeling and dynamics studies of proteins. J. Phys. Chem. B., 102:3586-3616, 1998.
Mackerell, Empirical force fields for biological macromolecules: overview and issues. J. Comput. Chem., 25(13):1584-604, 2004.
Maniatis et al., Molecular Cloning, A Laboratory Manual, Cold Spring Harbor Press, Cold Spring Harbor, N.Y., 1988.
Martyna et al., Remarks on “Constant-temperature molecular dynamics with momentum conservation.” Phys. Rev. E. Stat. Phys. Plasmas Fluids Relat. Interdiscip. Topics, 50(4):3234-3236, 1994.
Mellor et al., Gene, 24:1-14, 1983.
Mouneimne et al., Stable rightward shifts of the oxyhemoglobin dissociation curve induced by encapsulation of inositol hexaphosphate in red blood cells using electroporation, FEBS, 275:117-120, 1990.
Nicolau et al., Incorporation of Allosteric Effectors of Hemoglobin in Red Blood Cells. Physiologic Effects, Biblthca. Haemat., 51:92-107, 1985.
Offman et al., Rational engineering of L-asparaginase reveals importance of dual activity for cancer cell toxicity. Blood, 117(5):1614-1621, 2011.
Ollenschläger et al., Asparaginase-induced derangements of glutamine metabolism: the pathogenetic basis for some drug-related side-effects. Eur. J. Clin. Invest., 18(5):512-516, 1988.
Ortega et al., L-Asparaginase, vincristine, and prednisone for induction of first remission in acute lymphocytic leukemia. Cancer Res., 37(2):535-540, 1977.
Palm et al., A covalently bound catalytic intermediate in Escherichia coli asparaginase: crystal structure of a Thr-89-Val mutant. FEBS Lett., 390(2):211-216, 1996.
Penttila et al., Gene, 61:155-164, 1987.
Phillips et al., Scalable molecular dynamics with NAMD. J. Comput. Chem., 26(16):1781-1802, 2005.
Purwaha et al., Targeted metabolomic analysis of amino acid response to L-asparaginase in adherent cells. Metabolomics, 10:909-919, 2014.
Qin et al., Proc. Natl. Acad. Sci. USA, 95(24):14411-14416, 1998.
Reinert et al., Role of glutamine depletion in directing tissue-specific nutrient stress responses to L-asparaginase. J. Biol. Chem., 281(42):31222-31233, 2006.
Remington's Pharmaceutical Sciences, 18th Ed. Mack Printing Company, 1289-1329, 1990.
Sanches et al., Structural comparison of Escherichia coli L-asparaginase in two monoclinic space groups. Acta Crystallogr. D. Biol. Crystallogr., 59(Pt 3):416-422, 2003.
Scherf et al., A gene expression database for the molecular pharmacology of cancer. Nat. Genet., 24(3):236-244, 2000.
Sibakov et al., Eur. J. Biochem., 145:567 572, 1984.
Silverman et al., Improved outcome for children with acute lymphoblastic leukemia: results of Dana-Farber Consortium Protocol 91-01. Blood, 97(5):1211-1218, 2001.
Su et al., Correlation between asparaginase sensitivity and asparagine synthetase protein content, but not mRNA, in acute lymphoblastic leukemia cell lines. Pediatr. Blood Cancer, 50(2):274-279, 2008.
Swain et al., Crystal structure of Escherichia coli L-asparaginase, an enzyme used in cancer therapy. Proc. Natl. Acad. Sci. USA, 90(4):1474-1478, 1993.
Szymanska et al., Pharmacokinetic modeling of an induction regimen for in vivo combined testing of novel drugs against pediatric acute lymphoblastic leukemia xenografts. PLoS ONE. 7(3):e33894, 2012.
Verma et al., E. coli K-12 asparaginase-based asparagine biosensor for leukemia. Artif. Cells Blood Substit. Immobil. Biotechnol., 35(4):449-456, 2007.
Villa et al., L-asparaginase effects on inhibition of protein synthesis and lowering of the glutamine content in cultured rat hepatocytes. Toxicol. Lett., 32(3):235-241, 1986.
Ward, Proc, Embo-Alko Workshop on Molecular Biology of Filamentous Fungi, Helsinki, 119-128, 1989.
Warrell et al., Phase I evaluation of succinylated Acinetobacter glutaminase-asparaginase in adults. Cancer Res., 40(12):4546-4551, 1980.
Warrell et al., Clinical evaluation of succinylated Acinetobacter glutaminase-asparaginase in adult leukemia. Cancer Treat. Rep., 66(7):1479-1485, 1982.
Wawrzynczak and Thorpe, In: Immunoconjugates, Antibody Conuugates In Radioimaging And Therapy Of Cancer, Vogel (Ed.), NY, Oxford University Press, 28, 1987.
Wehner et al., Site-specific mutagenesis of Escherichia coli asparaginase II. None of the three histidine residues is required for catalysis. Eur. J. Biochem., 208(2):475-480, 1992.
Wu et al., Mechanism of sensitivity of cultured pancreatic carcinoma to asparaginase. Int. J. Cancer, 22(6):728-733, 1978.

1. An isolated, modified L-asparaginase (L-ASP) enzyme having at least one substitution relative to a native L-ASP amino acid sequence (see SEQ ID NO: 9), said at least one substitution including a glycine, threonine, or alanine at position 58 or a leucine or phenylalanine at position 59 of the native L-ASP sequence.

2. The enzyme of claim 1, wherein said at least one substitution comprises a leucine at position 59 of the native L-ASP sequence.

3. The enzyme of claim 1, wherein said at least one substitution comprises a phenylalanine at position 59 of the native L-ASP sequence.

4. The enzyme of claim 1, wherein said at least one substitution comprises a glycine at position 58 of the native L-ASP sequence.

5. The enzyme of claim 1, wherein said at least one substitution comprises a threonine at position 58 of the native L-ASP sequence.

6. The enzyme of claim 1, wherein said at least one substitution comprises an alanine at position 58 of the native L-ASP sequence.

7. The enzyme of claim 1, further comprising a heterologous peptide segment.

8. The enzyme of claim 1, wherein the enzyme is coupled to polyethylene glycol (PEG).

9. A nucleic acid comprising a nucleotide sequence encoding the enzyme of claim 1.

10. The nucleic acid of claim 9, wherein the nucleic acid is codon optimized for expression in bacteria, fungus, insects, or mammals.

11. An expression vector comprising the nucleic acid of either claim 9 or 10.

12. A host cell comprising the nucleic acid of either claim 9 or 10.

13. The host cell of claim 12, wherein the host cell is a bacterial cell, a fungal cell, an insect cell, or a mammalian cell.

14. A pharmaceutical formulation comprising the enzyme of claim 1 or the nucleic acid of either claim 9 or 10 in a pharmaceutically acceptable carrier.

15. A method of treating a tumor cell or subject having a tumor cell comprising administering to the tumor cell or the subject a therapeutically effective amount of the formulation of claim 14.

16. The method of claim 15, wherein the subject has been determined to have an ASNS-negative cancer.

17. The method of claim 15, wherein the subject is a human patient.

18. The method of claim 15, wherein the formulation is administered intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctival, intravesicularlly, mucosally, intrapericardially, intraumbilically, orally, by inhalation, by injection, by infusion, by continuous infusion, by localized perfusion bathing target cells directly, via a catheter, or via a lavage.

19. The method of claim 15, further comprising administering at least a second anticancer therapy to the subject.

20. The method of claim 19, wherein the second anticancer therapy is a surgical therapy, chemotherapy, radiation therapy, cryotherapy, hormone therapy, immunotherapy or cytokine therapy.

21. An isolated, modified L-asparaginase (L-ASP) enzyme having at least one substitution relative to a native L-ASP amino acid sequence (see SEQ ID NO: 9), said at least one substitution including a histidine at position 59 of the native L-ASP sequence.

22. The enzyme of claim 21, further comprising a heterologous peptide segment.

23. The enzyme of claim 21, wherein the enzyme is coupled to polyethylene glycol (PEG).

24. A nucleic acid comprising a nucleotide sequence encoding the enzyme of claim 21.

25. A pharmaceutical formulation comprising the enzyme of claim 21 or the nucleic acid of claim 24 in a pharmaceutically acceptable carrier.

26. A method of treating a tumor cell or subject having a tumor cell comprising administering to the tumor cell or the subject a therapeutically effective amount of the formulation of claim 25.

27. The method of claim 26, wherein the subject has been determined to have an ASNS-positive cancer.

28. The method of claim 26, wherein the subject is a human patient.

29. The method of claim 26, wherein the formulation is administered intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctival, intravesicularlly, mucosally, intrapericardially, intraumbilically, orally, by inhalation, by injection, by infusion, by continuous infusion, by localized perfusion bathing target cells directly, via a catheter, or via a lavage.

30. A composition comprising an enzyme according to either claim 1 or 21 or a nucleic acid according to either claim 9 or 24, for use in the treatment of a tumor cell in a subject.

31. The composition of claim 30, wherein the enzyme is coupled to polyethylene glycol (PEG).

32. The composition of claim 30, wherein the nucleic acid is codon optimized for expression in bacteria, fungus, insects, or mammals.

33. The composition of claim 30, wherein the composition is formulated for intratumoral, intravenous, intradermal, intraarterial, intraperitoneal, intralesional, intracranial, intraarticularly, intraprostatic, intrapleural, intratracheal, intraocular, intranasal, intravitreal, intravaginal, intrarectal, intramuscular, subcutaneous, subconjunctival, intravesicularl, mucosal, intrapericardial, intraumbilical, oral administration.

34. The composition of claim 30, further comprising at least a second anticancer therapy.

35. The composition of claim 34, wherein the second anticancer therapy is chemotherapy, hormone therapy, immunotherapy or cytokine therapy.

36. Use of an enzyme according to either claim 1 or 21 or a nucleic acid according to either claim 9 or 24 in the manufacture of a medicament for the treatment of a tumor cell.
